(19) United States
(12) Patent Application Publication
(10) Pub. No.: US 2011/0150770 A1
(43) Pub. Date: Jun. 23, 2011

(54) MULTIVALENT VACCINE AGAINST PORCINE TESCHOVIRUS AND OTHER DISEASE CAUSING ORGANISMS IN SWINE

(75) Inventors: Elida BAUTISTA, St. Joseph, MO (US); Dianna M. Murphy JORDAN, Ames, IA (US); Axel NEUBAUER, St. Joseph, MO (US); Abby Rae PATTERSON, Story City, IA (US); Michael B. Roof, Ames, IA (US); Eric M. VAUGHN, Ames, IA (US); Joseph G. Victoria, Ames, IA (US)

(73) Assignee: BOEHRINGER INGELHEIM VETMEDICA, INC., St. Joseph, MO (US)

(21) Appl. No.: 12/964,073

(22) Filed: Dec. 9, 2010

Related U.S. Application Data
(60) Provisional application No. 61/288,145, filed on Dec. 18, 2009.

(51) Int. Cl. A61K 39/295; A61K 39/39; A61K 39/125; A61K 49/00; A61P 31/04; A61P 31/14; B65D 85/00

(52) U.S. Cl. 424/9.2; 424/202.1; 424/201.1; 424/216.1; 206/570

(57) ABSTRACT

An immunogenic composition or vaccine, and method of treatment are provided by the present invention. The immunogenic composition is useful for treating, preventing, and lessening the severity of clinical symptoms associated with disease-causing organisms in swine, utilizing one or more Porcine Teschovirus antigen(s) along with an antigen of the other disease-causing organism in swine and a pharmaceutically acceptable carrier.

### SEQUENCE LISTING

The instant application contains a Sequence Listing which has been submitted in ASCII format via EFS-Web and is hereby incorporated by reference in its entirety. Said ASCII copy, created on Nov. 23, 2010, is named 10-0125-SEQ.txt and is 20,842 bytes in size.

### BACKGROUND

Teschovirus encephalomyelitis, which was previously known as Teschen disease, was first described as a particularly virulent encephalomyelitis of pigs. The disease was known to be highly fatal and was caused by strains of porcine teschovirus serotype 1 (PTV-1) of the genus Teschovirus, family Picornaviridae.

Talfan disease was identified as a less severe form of Teschovirus, also known as poliomyelitis suum or benign enzootic paresis. PTV has several serotypes including PTV 1, 2, 3, 4, 5, 6, 7, 8, 9, 10 and 11.

Picornaviruses comprise the genera Aphtovirus, Cardiovirus, Enterovirus, Erbovirus, Hepatovirus, Kobuvirus, Parechovirus, Rhinovirus, and Teschovirus. Picornaviruses are small enveloped, positive-stranded RNA viruses.

### SUMMARY OF INVENTION

It has been surprisingly found that Porcine Teschovirus (PTV) exacerbates other symptoms of disease-causing pathogens in swine, such that the severity of the symptoms are greatly increased when the pathogen is present with PTV infection. Thus, the immunogenic composition of the present invention has the surprising effect of reducing the symptoms of the pathogen, other than PTV, when administered. The present invention is directed towards an immunogenic composition or vaccine comprising one or more Porcine Teschovirus antigens and another non-Porcine Teschovirus disease-causing organism in swine.

The pathogenic effect of Porcine Teschovirus is reduced when the immunogenic composition of the present invention is administered to an animal. Animals administered the immunogenic composition of the present invention are at lower risk of developing clinical signs associated with Porcine Teschovirus than those animals not receiving any vaccine, after being infected with Porcine Teschovirus or receiving a vaccine not in accordance with the present invention.

The immunogenic composition of the present invention is effective for providing an immune response in an animal. Further, the immunogenic composition is effective for increasing an immune response in an animal when compared with those animals not receiving a vaccine The incidence of symptoms associated with Porcine Teschovirus is reduced in those animals administered the immunogenic composition. The present invention has the beneficial effect of reducing the incidence of symptoms associated with Porcine Teschovirus and the other disease causing organism which is part of the immunogenic composition. The immunogenic composition of the present invention advantageously reduces the exacerbation of symptoms of the other disease causing organism, when infection of PTV is found along with the infection of another disease causing organism.

The invention also provides for a method of treating or lessening the incidence of up to and including preventing porcine respiratory disease complex (PRDC) or post-weaning multisystemic wasting syndrome (PMWS) in a subject in need thereof, where the method comprises administering to the subject a therapeutically effective amount of an immunogenic composition comprising one or more porcine teschovirus antigens and a pharmaceutically acceptable carrier.

### BRIEF DESCRIPTION OF THE FIGURES

FIG. 1: Comparison of total clinical scores for pigs in each study group;

FIG. 2: Comparison of lung scores for pigs in each study group;

FIG. 3: Comparison Serological responses in conventional pigs—Anti-PRRSV ELISA;

FIG. 4: Average rectal temperatures in conventional pigs;

FIG. 5: Average clinical respiratory scores;

FIG. 6: Percentage of lung lesions for each of the conventional pigs;

FIG. 7: Average daily weight gain in conventional pigs;

FIG. 8: Antibody response to PRRSV for Groups 1 and 3;

FIG. 9: Comparison of anti-PTV antibody responses in conventional and CDCD pigs;

FIG. 10: PTV genome map;

FIG. 11: Average Rectal Temperature;

FIG. 12: Incidence of Pigs with Diarrhea;

FIG. 13: Incidence of Clinical Respiratory Signs;

FIG. 14: Percentage of lung lesions;

FIG. 15: Mean body weight;

FIG. 16: Average daily weight gain (ADWG);

FIG. 17: Macroscopic Lung Lesions;

FIG. 18: Macroscopic Lung Lesions.

### DETAILED DESCRIPTION OF THE PREFERRED EMBODIMENTS

### Definitions

“Clinical signs, symptoms and microscopic lesions of Porcine Teschovirus” are selected from, but not limited to, respiratory signs (coughing, tachypnea, dyspnea, clinical pneumonia), anorexia, pyrexia, lethargy, locomotor ataxia, agalatica, reproductive failure (increases in stillbirth fetuses, mummified fetuses, embryonic death, infertility), diarrhea, progressive weight loss, reduced weight gain, mortality, nonsuppurative polioencephalomyelitis with lymphocytic perivascular cuffs, neuronal degeneration, gliosis, hepatitis, myocarditis, interstitial pneumonia and combinations thereof.

“Clinical signs, symptoms and microscopic lesions of PRRS Virus”, include, but are not limited to, inappetence, pyrexia, abortions, transient discoloration or cyanosis of the ears, respiratory signs (dyspnea, tachypnea, coughing, clinical pneumonia), reluctance to drink, agalactia, mastitis, lethargy, very weak piglets at birth, reproductive failure (increases in abortions, stillbirth fetuses, mummified fetuses, embryonic death, early farrowing, prolonged anoestrus, delayed return to heat post-weaning reduced fertility), diarrhea, increase in secondary respiratory infections, such as Haemophilus parasuis and Streptococcus suis, rough hair coat, progressive weight loss, reduced weight gain, mortality, ocular discharge, interstitial pneumonia, lymphoplasmacytic perivasculitis, and combinations thereof.

“Clinical signs, symptoms and microscopic lesions of M. hyo” include, but are not limited to, respiratory signs (severe acute clinical pneumonia, coughing, dyspnea, tachypnea), pyrexia, high mortality, lymphoplasmacytic peribronchiolar cuffing, necrotizing bronchiolitis, and an increase in secondary bacterial respiratory infections.

“Clinical signs, symptoms and microscopic lesions of PCV2” include, but are not limited to, cutaneous pallor or icterus, progressive weight loss, reduced weight gain, mortality, diarrhea, pyrexia, respiratory signs (dyspnea, coughing, clinical pneumonia), reproductive disorders (abortion, stillbirths, mummies), interstitial pneumonia with interlobular edema, hepatitis, nephritis, myocarditis, lymphoplasmacytic perivasculitis, and enteritis.

“Clinical signs, symptoms and lesions of porcine respiratory disease complex (PRDC)” are selected from, but not limited to, respiratory signs (coughing, sneezing, dyspnea, tachypnea, clinical pneumonia), pyrexia, lethargy, anorexia, decreased weight gain, interstitial or bronchopneumonia.

“Clinical signs, symptoms and lesions of Post-weaning Multisystemic Wasting Syndrome (PMWS)” are selected from, but not limited to, wasting, paleness of the skin, unthriftiness, respiratory signs (coughing, dyspnea, tachypnea, clinical pneumonia), diarrhea, icterus, interstitial pneumonia with interlobular edema, hepatitis, nephritis, myocarditis, lymphoplasmacytic perivasculitis, and enteritis.

An “immunogenic or immunological composition” refers to a composition of matter that comprises at least one antigen, which elicits an immunological response in the host of a cellular and/or antibody-mediated immune response to the composition or vaccine of interest. Usually, an “immunological response” includes but is not limited to one or more of the following effects: the production or activation of antibodies, B cells, helper T cells, suppressor T cells, and/or cytotoxic T cells and/or gamma-delta T cells, directed specifically to an antigen or antigens included in the composition or vaccine of interest. Preferably, the host will display either a therapeutic or protective immunological response such that resistance to new infection will be enhanced and/or the clinical severity of the disease reduced. Such protection will be demonstrated by either a reduction or lack of clinical signs normally displayed by an infected host, a quicker recovery time and/or a lowered duration or viral titer in the tissues or body fluids or excretions of the infected host.

“Mortality”, in the context of the present invention, refers to death caused by an infection of PTV or another non-PTV pathogen. This includes the situation where the infection is so severe that an animal is euthanized to prevent suffering and provide a humane ending to their life.

“Attenuation” means reducing the virulence of a pathogen. In the present invention “attenuation” is synonymous with “avirulent”.

An “effective amount” for purposes of the present invention, means an amount of an immunogenic composition capable of inducing an immune response that reduces the incidence of or lessens the severity of infection in an animal. Particularly, an effective amount refers to colony forming units (CFU) per dose.

The term “in need of such administration” or “in need of such administration treatment”, as used herein means that the administration/treatment is associated with the boosting or improvement in health or any other positive medicinal effect on health of the animals which receive the immunogenic composition in accordance with the present invention.

The term “subunit immunogenic composition” as used herein refers to a composition containing at least one immunogenic polypeptide or antigen, but not all antigens, derived from or homologous to an antigen from a given pathogen. Such a composition is substantially free of the intact pathogen. Thus, a “subunit immunogenic composition” is prepared from at least partially purified or fractionated (preferably substantially purified) immunogenic polypeptides from a pathogen, or recombinant analogs thereof.

The terms “immunogenic protein” or “immunogenic component or polypeptide or antigen” as used herein refer to an amino acid sequence which elicits an immunological response as described above. An “immunogenic” component or antigen, as used herein, includes the full-length sequence of any proteins of a given pathogen, analogs thereof, or immunogenic fragments thereof. The term “immunogenic fragment” refers to a fragment of a protein which includes one or more epitopes and thus elicits the immunological response described above. Such fragments can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, N.J. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. USA 81:3998-4002; Geysen et al. (1986) Molec. Immunol. 23:709-715. Similarly, conformational epitopes are readily identified by determining spatial conformation of amino acids such as by, e.g., x-ray crystallography and 2-dimensional nuclear magnetic resonance. See, e.g., Epitope Mapping Protocols, supra, all of which are incorporated by reference.

Synthetic antigens are also included within the definition, for example, polyepitopes, flanking epitopes, and other recombinant or synthetically derived antigens. See, e.g., Bergmann et al. (1993) Eur. J. Immunol. 23:2777-2781; Bergmann et al. (1996), J. Immunol. 157:3242-3249; Suhrbier, A. (1997), Immunol. and Cell Biol. 75:402-408; Gardner et al., (1998) 12th World AIDS Conference, Geneva, Switzerland, Jun. 28-Jul. 3, 1998, all incorporated by reference.

“Sequence Identity” as it is known in the art refers to a relationship between two or more polypeptide sequences or two or more polynucleotide sequences, namely a reference sequence and a given sequence to be compared with the reference sequence. Sequence identity is determined by comparing the given sequence to the reference sequence after the sequences have been optimally aligned to produce the highest degree of sequence similarity, as determined by the match between strings of such sequences. Upon such alignment, sequence identity is ascertained on a position-by-position basis, e.g., the sequences are “identical” at a particular position if at that position, the nucleotides or amino acid residues are identical. The total number of such position identities is then divided by the total number of nucleotides or residues in the reference sequence to give % sequence identity. Sequence identity can be readily calculated by known methods, including but not limited to, those described in Computational Molecular Biology, Lesk, A. N., ed., Oxford University Press, New York (1988), Biocomputing: Informatics and Genome Projects, Smith, D. W., ed., Academic Press, New York (1993); Computer Analysis of Sequence Data, Part I, Griffin, A. M., and Griffin, H. G., eds., Humana Press, New Jersey (1994); Sequence Analysis in Molecular Biology, von Heinge, G., Academic Press (1987); Sequence Analysis Primer, Gribskov, M. and Devereux, J., eds., M. Stockton Press, New York (1991); and Carillo, H., and Lipman, D., SIAM J. Applied Math., 48: 1073 (1988), the teachings of which are incorporated herein by reference.

Preferred methods to determine the sequence identity are designed to give the largest match between the sequences tested. Methods to determine sequence identity are codified in publicly available computer programs which determine sequence identity between given sequences. Examples of such programs include, but are not limited to, the GCG program package (Devereux, J., et al., Nucleic Acids Research, 12(1):387 (1984)), BLASTP, BLASTN and FASTA (Altschul, S. F. et al., J. Molec. Biol., 215:403-410 (1990). The BLASTX program is publicly available from NCBI and other sources (BLAST Manual, Altschul, S. et al., NCVI NLM NIH Bethesda, Md. 20894, Altschul, S. F. et al., J. Molec. Biol., 215:403-410 (1990), the teachings of which are incorporated herein by reference). These programs optimally align sequences using default gap weights in order to produce the highest level of sequence identity between the given and reference sequences.

As an illustration, by a polynucleotide having a nucleotide sequence having at least, for example, 85%, preferably 90%, even more preferably 95% “sequence identity” to a reference nucleotide sequence, it is intended that the nucleotide sequence of the given polynucleotide is identical to the reference sequence except that the given polynucleotide sequence may include up to 15, preferably up to 10, even more preferably up to 5 point mutations per each 100 nucleotides of the reference nucleotide sequence. In other words, in a polynucleotide having a nucleotide sequence having at least 85%, preferably 90%, even more preferably 95% identity relative to the reference nucleotide sequence, up to 15%, preferably 10%, even more preferably 5% of the nucleotides in the reference sequence may be deleted or substituted with another nucleotide, or a number of nucleotides up to 15%, preferably 10%, even more preferably 5% of the total nucleotides in the reference sequence may be inserted into the reference sequence.

These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence. Analogously, by a polypeptide having a given amino acid sequence having at least, for example, 85%, preferably 90%, even more preferably 95% sequence identity to a reference amino acid sequence, it is intended that the given amino acid sequence of the polypeptide is identical to the reference sequence except that the given polypeptide sequence may include up to 15, preferably up to 10, even more preferably up to 5 amino acid alterations per each 100 amino acids of the reference amino acid sequence. In other words, to obtain a given polypeptide sequence having at least 85%, preferably 90%, even more preferably 95% sequence identity with a reference amino acid sequence, up to 15%, preferably up to 10%, even more preferably up to 5% of the amino acid residues in the reference sequence may be deleted or substituted with another amino acid, or a number of amino acids up to 15%, preferably up to 10%, even more preferably up to 5% of the total number of amino acid residues in the reference sequence may be inserted into the reference sequence.

These alterations of the reference sequence may occur at the amino or the carboxy terminal positions of the reference amino acid sequence or anywhere between those terminal positions, interspersed either individually among residues in the reference sequence or in the one or more contiguous groups within the reference sequence. Preferably, residue positions which are not identical differ by conservative amino acid substitutions. However, conservative substitutions are not included as a match when determining sequence identity.

“Sequence homology”, as used herein, refers to a method of determining the relatedness of two sequences. To determine sequence homology, two or more sequences are optimally aligned, and gaps are introduced if necessary. However, in contrast to “sequence identity”, conservative amino acid substitutions are counted as a match when determining sequence homology. In other words, to obtain a polypeptide or polynucleotide having 95% sequence homology with a reference sequence, 85%, preferably 90%, even more preferably 95% of the amino acid residues or nucleotides in the reference sequence must match or comprise a conservative substitution with another amino acid or nucleotide, or a number of amino acids or nucleotides up to 15%, preferably up to 10%, even more preferably up to 5% of the total amino acid residues or nucleotides, not including conservative substitutions, in the reference sequence may be inserted into the reference sequence. Preferably the homolog sequence comprises at least a stretch of 50, even more preferably at least 100, even more preferably at least 250, and even more preferably at least 500 nucleotides. A “conservative substitution” refers to the substitution of an amino acid residue or nucleotide with another amino acid residue or nucleotide having similar characteristics or properties including size, hydrophobicity, etc., such that the overall functionality does not change significantly.

### PTV Immunogenic Compositions and Vaccines

The immunogenic composition of one or more strains of antigens of Porcine Teschovirus (PTV) is effective for providing an immune response in an animal. Further, the immunogenic composition is effective for increasing an immune response in an animal when compared with those animals not receiving a vaccine. Thus, the present invention is directed towards an immunogenic composition or vaccine comprising one or more PTV antigens in swine. Preferably, the immunogenic composition comprises one or more antigens of Porcine Teschovirus and optionally a pharmaceutically acceptable carrier.

The pathogenic effect of PTV is reduced when the immunogenic composition of the present invention is administered to an animal. Animals administered the immunogenic composition of the present invention are at lower risk of developing clinical signs associated with PTV than those animals not receiving any vaccine, after being infected with PTV or receiving a vaccine not in accordance with the present invention.

The present invention also provides for strains of PTV, which can be attenuated, inactivated, an immunogenic subunit, a DNA sequence coding for a PTV antigen, a plasmid containing PTV DNA sequences therein, and combinations thereof. The genome map of PTV is shown as FIG. 10. Any of the PTV subunits VP4, VP2, VP3, VP1, 2A, 2B, 2C, 3A, 3B, 3C, 3D or combinations thereof may be used in the immunogenic composition. Preferably, the PTV utilized in the embodiments of the present invention are selected from above, however, any PTV strain will work for purposes of the present invention.

Suitable sequences of PTV include, but are not limited to, GenBank Accession Nos. AF231769, AF296096, AF296088, AF296095, AF296094, AF296090, AF296089, AF296093, AF296093, AF296091, AF296087, PTVBIVI and combinations thereof. PTV serotype 2 and PTVBIVI are more preferred, and PTVBIVI is most preferred as the PTV antigen used in the present invention. The full length sequence of PTVBIVI is embodied as SEQ ID NO:1 or SEQ ID NO: 2 and sequence homologues of 95%, 90%, 85%, 80%, 75% or 70% are most preferred.

### Multi-Valent Immunogenic Compositions and Vaccines

It has been found that Porcine Teschovirus (PTV) exacerbates other symptoms of disease-causing pathogens in swine, such that the severity of the symptoms are increased when the pathogen is present with PTV infection. Thus, the immunogenic composition of the present invention has the surprising effect of reducing the symptoms of the non-PTV pathogen(s) when administered. Thus, the present invention is directed towards an immunogenic composition or vaccine comprising one or more PTV antigens and another non-PTV disease-causing organism in swine.

In preferred forms of the present invention, the immunogenic composition comprises one or more Porcine Teschovirus antigens, at least one immunogenic component effective against another disease-causing organism other than Porcine Teschovirus, and a pharmaceutically acceptable carrier.

Additionally, herds would experience a smaller number of infected or deceased animals in a herd when animals are administered the vaccine in accordance with the present invention, when compared to non-vaccinated, but infected animals, and, preferably even as compared to animals vaccinated with conventionally available vaccine(s), as it has been determined that the PTV is highly infectious and transmissible.

The incidence of symptoms associated with Porcine Teschovirus is reduced in those animals administered the immunogenic composition. The present invention has the beneficial effect of reducing the incidence of symptoms associated with Porcine Teschovirus and the other disease causing organism which is part of the immunogenic composition. The immunogenic composition of the present invention advantageously reduces the exacerbation of symptoms of the other disease causing organism, when infection of PTV is found along with the infection of another disease causing organism.

The antigen from the other disease-causing organism, for purposes of the present invention, can be from any pathogen affecting swine. Preferably, the non-PTV or other disease-causing organism is Actinobacillus pleuropneumonia; Haemophilus parasuis preferably subtypes 1, 7 and 14, Mycoplasma hyopneumoniae (M. hyo.), Porcine circovirus-2 (PCV-2), Porcine Reproductive and Respiratory Syndrome (PRRS) Virus, Reovirus, or Swine Influenza Virus (SIV). In a most preferred embodiment, the other disease-causing organism is PCV-2, PRRS Virus, M. hyo and combinations thereof.

Preferably, the PCV2 immunogenic component of the immunogenic composition is selected from the group consisting of attenuated PCV2, inactivated PCV2, an immunogenic subunit of PCV2, a plasmid containing PCV2 DNA sequences therein, and combinations thereof. In a most preferred embodiment, the PCV2 component is an ORF2 PCV2 protein or an immunogenic fragment thereof.

Preferably, the PRRS Virus component of the vaccine is selected from the group consisting of attenuated PRRSV, inactivated PRRSV, an immunogenic subunit of PRRSV, a plasmid containing PRRSV DNA sequences therein, and combinations thereof.

The invention also provides for a method of treating or lessening the incidence of up to and including preventing porcine respiratory disease complex (PRDC) or post-weaning multisystemic wasting syndrome (PMWS) in a subject in need thereof, where the method comprises administering to the subject a therapeutically effective amount of an immunogenic composition comprising one or more porcine teschovirus antigens and a pharmaceutically acceptable carrier.

In a preferred embodiment, the method further comprises administration of an antigen of a further disease-causing pathogen in swine, other than Porcine Teschovirus. Preferably, the subject exhibits clinical signs of PRDC, PMWS, or combination thereof.

In some embodiments of the present invention, the clinical symptoms of PRDC or PMWS are also associated with Mycoplasma hyopneumoniae (M. hyo) infection. The method of the present invention is effective for reducing the incidence of or severity of clinical signs of the other pathogen to a greater extent than administration of the immunogenic component administered in the absence of administration of Porcine Teschovirus antigen.

The present invention also provides for a method of producing an immunogenic composition or vaccine comprising the steps of providing at least one Porcine Teschovirus antigen; providing at least one immunogenic component effective against another disease-causing organism other than Porcine Teschovirus; and combining the first two components with a pharmaceutically acceptable carrier. Preferably, the immunogenic component effective against another disease-causing organism other than Porcine Teschovirus is selected from Actinobacillus pleuropneumonia; Haemophilus parasuis, preferably subtypes 1, 7 and 14; Mycoplasma hyopneumoniae (M. hyo); Porcine circovirus-2 (PCV-2); Porcine Reproductive and Respiratory Syndrome (PRRS) Virus; Reovirus; Swine Influenza Virus (SIV), and combinations thereof.

The present invention additionally provides for a method of reducing the incidence of or severity of one or more clinical signs associated with porcine respiratory disease complex or postweaning multisystem wasting syndrome in a subject, wherein the method comprises the step of administering the immunogenic composition of the present invention to a subject in need thereof, and wherein the reduction of the incidence of or the severity of the one or more clinical signs is relative to a subject not receiving the immunogenic composition.

The present invention further provides for a method for evaluating the ability of an immunogenic composition to prevent or reduce the severity of PRDC or PMWS in a porcine subject, where the method comprises the steps of administering to the subject a candidate immunogenic composition; exposing the subject to a Porcine Teschovirus isolate in an amount sufficient to cause infection in an unvaccinated subject; and monitoring the subject for one or more clinical signs of PRDC or PMWS, thereby evaluating the ability of the candidate immunogenic composition to prevent or reduce the severity of PRDC or PMWS.

### Kits

In other forms of the present invention, a kit is provided wherein the kit comprises (i) one or more antigens of Porcine Teschovirus; (ii) at least one immunogenic component effective against another disease-causing organism other than Porcine Teschovirus; (iii) a pharmaceutically acceptable carrier; and (iv) a container for packaging said antigens and said pharmaceutically acceptable carrier. The immunogenic component of another disease-causing organism is preferably selected from Actinobacillus pleuropneumonia; Haemophilus parasuis, Mycoplasma hyopneumoniae (M. hyo.), Porcine circovirus-2 (PCV-2), Porcine Reproductive and Respiratory Syndrome (PRRS) Virus, Reovirus, or Swine Influenza Virus (SIV), and combinations thereof.

### Administration

The composition of the present invention can be administered in any conventional manner. Examples of administration methods include any method that affords access by cells of the immune system to the immunogenic composition: transdermal, intradermal, intratracheal, intragastrical, intravaginal, intrarectal, intramuscular, intranasal, intravenous, direct injection into target tissues, intraarterial, intraperitoneal, oral, intrathecal, subcutaneous, intracutaneous, intracardial, intralobal, intramedullar, intrapulmonary, and combinations thereof. Preferred modes of administration are intramuscular, subcutaneous and intranasal, with subcutaneous and intranasal being especially preferred.

If desired or necessary, booster immunizations may be given once or several times at various intervals. However, it is a preferred embodiment of the present invention that the vaccination be administered as a single-dose.

After administration of such a vaccine, an immune response is elicited in the animal and signs of Porcine Teschovirus infection or infection of other pathogen affecting swine, other than Porcine Teschovirus, are reduced in incidence and/or severity, as well as a reduction in rate of mortality, in comparison to animals exposed to wild-type bacteria or isolates after challenge with a virulent form of Porcine Teschovirus or other pathogen affecting swine.

### Carriers and Adjuvants

A pharmaceutically-acceptable carrier or adjuvant may be present in the immunogenic composition. The adjuvant can be any adjuvant suitable for use in a pharmacological composition. In one preferred embodiment, the immunogenic composition of the present invention contains an adjuvant. “Adjuvants” as used herein, can include aluminum hydroxide and aluminum phosphate, saponins e.g., Quil A, QS-21 (Cambridge Biotech Inc., Cambridge Mass.), GPI-0100 (Galenica Pharmaceuticals, Inc., Birmingham, Ala.), water-in-oil emulsion, oil-in-water emulsion, water-in-oil-in-water emulsion. The emulsion can be based in particular on light liquid paraffin oil (European Pharmacopea type); isoprenoid oil such as squalane or squalene; oil resulting from the oligomerization of alkenes, in particular of isobutene or decene; esters of acids or of alcohols containing a linear alkyl group, more particularly plant oils, ethyl oleate, propylene glycol di-(caprylate/caprate), glyceryl tri-(caprylate/caprate) or propylene glycol dioleate; esters of branched fatty acids or alcohols, in particular isostearic acid esters. The oil is used in combination with emulsifiers to form the emulsion. The emulsifiers are preferably nonionic surfactants, in particular esters of sorbitan, of mannide (e.g. anhydromannitol oleate), of glycol, of polyglycerol, of propylene glycol and of oleic, isostearic, ricinoleic or hydroxystearic acid, which are optionally ethoxylated, and polyoxypropylene-polyoxyethylene copolymer blocks, in particular the Pluronic® products, especially L121 (commercially available from BASF). See Hunter et al., The Theory and Practical Application of Adjuvants (Ed.Stewart-Tull, D. E. S.). JohnWiley and Sons, NY, pp 51-94 (1995) and Todd et al., Vaccine 15:564-570 (1997), all hereby incorporated by reference.

For example, it is possible to use the SPT emulsion described on page 147 of “Vaccine Design, The Subunit and Adjuvant Approach” edited by M. Powell and M. Newman, Plenum Press, 1995, and the emulsion MF59 described on page 183 of this same book.

A further instance of an adjuvant is a compound chosen from the polymers of acrylic or methacrylic acid and the copolymers of maleic anhydride and alkenyl derivative. Advantageous adjuvant compounds are the polymers of acrylic or methacrylic acid which are cross-linked, especially with polyalkenyl ethers of sugars or polyalcohols. These compounds are known by the term carbomer (Phameuropa Vol. 8, No. 2, June 1996, incorporated by reference).

Persons skilled in the art can also refer to U.S. Pat. No. 2,909,462 (incorporated by reference) which describes such acrylic polymers cross-linked with a polyhydroxylated compound having at least 3 hydroxyl groups, preferably not more than 8, the hydrogen atoms of at least three hydroxyls being replaced by unsaturated aliphatic radicals having at least 2 carbon atoms. The preferred radicals are those containing from 2 to 4 carbon atoms, e.g. vinyls, allyls and other ethylenically unsaturated groups. The unsaturated radicals may themselves contain other substituents, such as methyl. The products sold under the name Carbopol® (BF Goodrich, Ohio, USA) are particularly appropriate. They are cross-linked with an allyl sucrose or with allyl pentaerythritol. Among then, there may be mentioned Carbopol 974P, 934P and 971P. Most preferred is the use of Cabopol 971P. Among the copolymers of maleic anhydride and alkenyl derivative, are the copolymers EMA (Monsanto), which are copolymers of maleic anhydride and ethylene. The dissolution of these polymers in water leads to an acid solution that will be neutralized, preferably to physiological pH, in order to give the adjuvant solution into which the immunogenic, immunological or vaccine composition itself will be incorporated.

Further suitable adjuvants include, but are not limited to, the RIBI adjuvant system (Ribi Inc.), Block co-polymer (CytRx, Atlanta Ga.), SAF-M (Chiron, Emeryville Calif.), monophosphoryl lipid A, Avridine lipid-amine adjuvant, heat-labile enterotoxin from E. coli (recombinant or otherwise), cholera toxin, IMS 1314 or muramyl dipeptide, or naturally occurring or recombinant cytokines or analogs thereof or stimulants of endogenous cytokine release, among many others.

Preferably, the adjuvant is added in an amount of about 100 μg to about 10 mg per dose. Even more preferably, the adjuvant is added in an amount of about 100 μg to about 10 mg per dose. Even more preferably, the adjuvant is added in an amount of about 500 μg to about 5 mg per dose. Even more preferably, the adjuvant is added in an amount of about 750 μg to about 2.5 mg per dose. Most preferably, the adjuvant is added in an amount of about 1 mg per dose.

Preferably, the pharmaceutically acceptable carrier is selected from the group consisting of solvents, dispersion media, coatings, stabilizing agents, diluents, preservatives, antibacterial and antifungal agents, isotonic agents, adsorption delaying agents, adjuvants, immune stimulants, and combinations thereof.

For purposes of the present invention, the subject is any animal or human susceptible to developing Porcine Teschovirus, including, but not limited to swine, bovine, deer, horses, canines, felines, mammals, birds, humans, or reptiles. Preferably, the subject is a mammal and more preferably a swine and among swine preferably, a young PTV-negative piglet, a barrier-raised specific pathogen-free piglet, or a caesarian-delivered piglet.

### Dosage

In preferred forms, the dose volume of the vaccine is no more than 5 ml, more preferably no more than 3 ml, and more preferably no more than 2 ml. In some preferred forms, a second or subsequent administration of the immunogenic composition would be administered after the first administration. Such a subsequent administration would preferably occur at least 10 days after the initial administration, more preferably between at least 10-32 days, more preferably between at least 12-30 days, still more preferably at least 14 days, and most preferably between at least 14-28 days. In most preferred forms, the vaccine would be administered either on Day 0 as a single dose, or, in alternative forms, on Day 0 and 14-28 days thereafter with exposure to pathogenic forms of Porcine Teschovirus not occurring until after the completion of the immunizing regimen. In a most preferred form, no booster is necessary and the vaccine is administered only one time. The vaccine is administered to animals from 1 day of age through adulthood, preferably from 1 day of age through 2 years of age, more preferably to pigs from 1 day of age through 16 weeks of age, and most preferably to pigs from 3 weeks to 12 weeks of age.

### Clinical Signs

The present invention also reduces clinical signs or symptoms of PTV and other pathogens affecting swine. Other pathogens are preferably PRRS, PCV2, M. Hyo and the clinical signs or symptoms of these pathogens are also reduced. Clinical signs or symptoms of porcine respiratory disease complex (PRDC) or Post-weaning Multisystemic Wasting Syndrome (PMWS) are also preferably reduced. Preferably, clinical signs are reduced at least 10%, more preferably, by at least 20%, even more preferably, by at least 25%, more preferably, by at least 30%, even more preferably, by at least 40%, still more preferably, by at least 50%, even more preferably, by at least 56%, still more preferably, by at least 60%, even more preferably, by at least 70%, still more preferably, by at least 75%, even more preferably, by at least 80%, still more preferably, by at least 83%, and, most preferably, by at least 90% as compared to those animals not receiving a vaccine.

The following examples are representative of preferred embodiments of the present invention. It is understood that nothing herein should be taken as a limitation upon the overall invention.

### Example 1

### Objective

(1) To fulfill Koch's postulates by determining whether the PTV viruses isolated by BI investigators from a swine disease outbreak investigation can cause central nervous system (CNS) signs in naïve animals.

(2) To determine whether concurrent exposure to porcine respiratory and reproductive syndrome virus (PRRSV) and porcine circovirus type 2 (PCV2) prior to exposure to PTV enhance CNS signs in naïve animals.

(3) To evaluate the effectiveness of an inactivated PTV vaccine to induce antigen-specific immune responses.

### Materials and Methods

### Experimental Design

To fulfill objectives 1-3, conventional pigs were used. The study consisted of 6 experimental groups (groups 1-6) and one control group (group 7). The animals in experimental groups 1-5 were infected with a total volume of 2 ml of the single or combined virus inoculum as indicated in Table 3. Experimental group 6 was inoculated with the indicated killed virus and the control group was shaminoculated with media. The composition and titer of the viral challenge materials are presented in Table 4. All animals were observed daily for abnormal clinical signs. All animals were bled at study days 0, 3, 7, 14, 21, 28, and 35. All animals were weighed at study day 0, 14 and 35. Two pigs from the experimental groups 1 to 5 and from the control group (group 7) were euthanized at study days 14, 21, and 28. All remaining pigs were euthanized at study day 35.

TABLE 3
Treatment Groups and Treatment

| | | Treatment | Treatment | | |
|---|---|---|---|---|---|
| Group # | n | Group | Day 0 | Day 7 | Day 14 |
| 1 | 11 | PRRS + PCV2 | PRRSV (IN) | MEM (IV) | None |
| 2 | 11 | PRRS + PCV2 + | PRRSV and | PTVa and | None |
| | | PTV | PCV2 (IN) | MEM (IV) | |
| 3 | 11 | PTV | MEM (IN) | PTVa (IV) | None |
| 4 | 11 | Mix PTV | MEM (IN) | PTVb-d (IV) | None |
| 5 | 11 | Reovirus | MEM (IN) | Reovirus | None |
| 6 | 6 | Killed PTV | kPTV (IM) | None | kPTV |
| | | vaccine | | | (IM) |
| 7 | 11 | Control | MEM (IN) | MEM (IV) | None |

Treatment groups where IN=intranasal; IV=intravenous; PRRSV=porcine reproductive and respiratory syndrome virus, BI internal reference strain 972-1; PCV2=Porcine circovirus type 2, BI internal reference strain 194-8, Reovirus=Internal BI reference strain Unk-BHK-6137C-5 isolate; PTV=porcine teschovirus, internal BI reference strain PTV-6137A-1 (a), PTV-PKWRL-968-1 (b), PTV-PK2a-969-2 (c), and PTV-ST-972-1 (d); kPTV=inactivated PTV, BI internal reference strain PTV-6137A-1 propagated in PK-WRL cells to pass 2 inactivated with BEI and formulated with Incomplete Freund's adjuvant; MEM=minimal essential media; n=number of animals/group; and None=no treatment given.

TABLE 4
Composition and titer of the viral challenge materials

| Virus | Titer | BI internal reference identification no. |
|---|---|---|
| PRRSV | 4.5 logs TCID₅₀/ml | sw0022208-972-1 |
| PCV2 | 4.0 logs TCID₅₀/ml | sw022208-194-8 |
| Reovirus | 5.0 logs TCID₅₀/ml | sw022208-6137C-5 |
| PTVa | 5.0 logs TCID₅₀/ml | sw022208-6137A-1 |
| PTVb | 5.0 logs TCID₅₀/ml | PTV sw022208-968-1 |
| PTVc | 4.0 logs TCID₅₀/ml | PTV sw022208-969-2 |
| PTVd | 4.0 logs TCID₅₀/ml | sw022208-972-1 |
| kPTV | 5.0 logs TCID₅₀/ml | sw022208-6137A-1 BEI inactivated and |
| | | formulated with Incomplete |
| | | Freund's adjuvant |

### Sample and Data Collection

### Whole Blood Collection

Blood was collected from conventional pigs on days −18, 0, 3 or 4, 7, 14, 21, 28, and 35 and separate aliquots for serology and virus isolation prepared.

### Weights

Animals were weighed at the initiation of the study (study day 0) and at necropsy date so that the animals' average daily weight gain (ADWG) could be determined.

### Clinical Observations

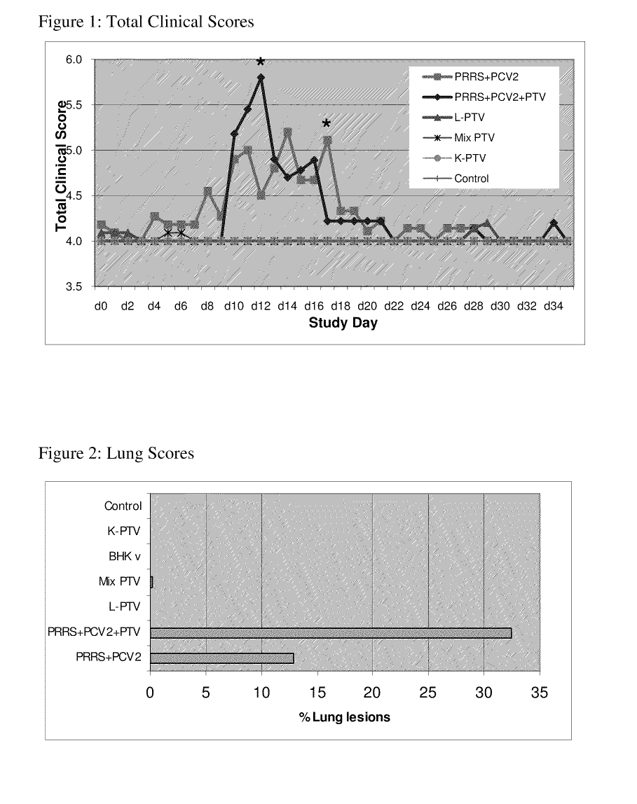

Animals were observed daily for clinical signs from day 0 to day 35. The total Clinical Scores and Lung Scores are shown in FIGS. 1 and 2. Special attention was placed to determine development of neurological abnormalities such as lack of coordination, tremors, sternal and/or lateral recumbency, convulsions, paralysis or inability to stand or walk, etc. in the infected pigs and or reactivity in the vaccinated pigs. Other clinical signs including respiratory signs and diarrhea were also noted and recorded. The clinical condition of these animals was evaluated based on a numerical index reflecting the severity of illness. Scores for each of the individual observations ranged from 1 to 3 with 1 assigned for a normal condition, 2 for mild condition and score of 3 for a severe condition. The total score consisted of the sum of the daily observations for each abnormal observation. A dead animal as a result of the infection was given a total score of 4 for each condition observed.

### Rectal Temperatures

Measurements were recorded daily from study day 0 to 14 and twice a week thereafter.

### Necropsy

The brain, spinal cord, tonsil, thymus, lung, heart, spleen, lymph nodes, spleen, liver, kidney and intestinal organs of the study animals were evaluated for evidence of gross lesions as compared to sham-infected control pigs. The percentage of lung lesions was determined and scored for each pig at necropsy and lesions in any other tissue were also recorded if present. Samples from brain, spinal cord, tonsil, thymus, lung, heart, spleen, lymph nodes, liver and intestinal tract were collected in 10% buffered formalin and submitted to ISU-VDL for histopathology analysis to determine microscopic lesions. Another set of samples that included brain, spinal cord, tonsil, lung, lymph nodes, and spleen were collected for virological evaluation. Tissue samples for virological analysis were collected and shipped in dry ice and/or stored at −70° C. until processing. Tissue homogenates were prepared in basal media as 5-10% suspension, clarified by centrifugation and filtered through 0.22 μm filters. Aliquots were stored at −70° C. until ready for analysis.

### Serological Evaluation

Serum samples were tested for antibodies to PRRSV, PCV2 and PTV. Antibodies to PRRSV were measured by IDEXX ELISA and reported as S/P ratios by the Boehringer Ingelheim Vetmedica, Inc. Health Management Center Diagnostic lab in Ames, Iowa. Antibodies to PCV2 were measured by indirect fluorescence antibody (IFA) test and antibody titers were reported as the mean of the reciprocal of last dilution with specific fluorescence. Antibodies to PTV were measured by virus neutralization assay (VNA) and a subset of samples was also tested by IFA. For PTV serology, the PTV-p6137A-1 isolate as used as antigen for the assay results reported in this study. The VNA was performed with heat-inactivated serum. Anti-PTV antibody titers were reported as the mean of the reciprocal of last dilution that neutralized virus-induced CPE for VNA or that showed specific florescence for IFA. Antibody responses in the animals exposed to the unidentified reovirus were also analyzed by IFA.

### Viral Assays

Serum and tissue homogenates were analyzed for virus isolation to confirm the recovery of infectious PRRSV, PCV2, PTV and the unidentified reovirus. Virus isolation was performed by inoculating two-day old monolayers prepared in 96-well plates of the following cell lines: AKMA104, VIDO-R1, PK/WRL, PK2a, ST BHK21 using approximately 20 ul sample/well and 4 wells per sample. Virus Isolation was confirmed based on CPE and immuno-fluorescence staining of pass 3 cultures with available virus-specific antibodies. PRRSV isolation was confirmed based on CPE in AK-MA1904 cells and staining with the SR-30 monoclonal anti-PRRSV antibody. PCV2 isolation was confirmed based on staining of pass 3 VIDO-R1 with anti-PCV2 ORF2 monoclonal antibody. PTV isolation was confirmed based on CPE in PKWRL, PK2a or ST cells and staining with a polyclonal swine anti-PTV/PEV antibody. The reovirus isolation was confirmed by PCR of pass 3 BHK21 cultures.

### Results

### Clinical Observations

Animals were observed daily for the duration of the study and monitored for the development of clinical signs that included neurological signs, mortality, respiratory disease, and diarrhea.

### Neurological Signs

No significant neurological signs were observed in animals singularly inoculated with PTV. One pig in group 1 that was infected with PRRSV and PCV2 developed more severe CNS and moderate respiratory signs at days 8 and 9 and was found dead at day 10. Five other pigs in this group showed some mild neurological signs at different study days: One pig at day 8, one pig at day 10, one pig at days 15 and 17, and two other pigs at day 17. In the group 2 that was infected with PRRSV+PCV2 and one week later with PTV, only one animal showed mild neurological signs at day 34. No neurological signs were recorded for any other pig in the study.

### Diarrhea

Diarrhea was observed in 1 or 2 pigs at study days 0, 1, 4-8, 10, 11, 13, 14, and 17 in the group 1 (infected with PRRSV+PCV2) for a total of 6 pigs affected during the study period. In group 2 (PRRSV+PCV2+PTV) only one animal had mild diarrhea at day 18. In group 3 (PTV) only one animal had mild diarrhea at days 1-3. In group 4 (mix PTV) one animal had mild diarrhea at days 5 and 6. In group 5 (Reovirus) two animals had mild diarrhea at day 1. None of the pigs in groups 6 and 7 had diarrhea during the study period.

### Respiratory Signs

Respiratory abnormalities were observed in all the pigs infected with PRRSV+PCV2 in groups 1 and 2. The clinical respiratory signs were first observed at day 8 in the pig that died on day 10 from group 1. At or on day 10 approximately 70% of the PRRSV+PCV2-exposed pigs showed respiratory signs. All the pigs in group 1 had only mild respiratory signs at various times of the study except for one pig at day 14. In contrast, in group 2a relatively high number of pigs developed severe respiratory signs at day 10 which persisted in the group until day 13: 5 pigs at day 10, 7 pigs at day 11, 8 pigs at day 12, and 3 pigs at day 13. In group 3 only one animal had mild respiratory signs at days 28 and 29. No respiratory signs were observed in any of the conventional pigs of groups 4, 5, 6 and 7. The level of severity of the respiratory signs was higher in the group 2 with the PRRSV+PCV2 exposed animals that were also infected with PTV than in the group 1 that was only infected with PRRSV+PCV2. The difference in the respiratory scores between these two groups as well as with the PTV only infected and control groups can be best appreciated in graph 2. Statistical analysis comparing the mean respiratory scores by T-test show a significant difference between group 1 and 2 at study days 11 and 12, which was confirmed by Kruskal-Wallis/Wilcoxon Two Sample Test with P<0.05.

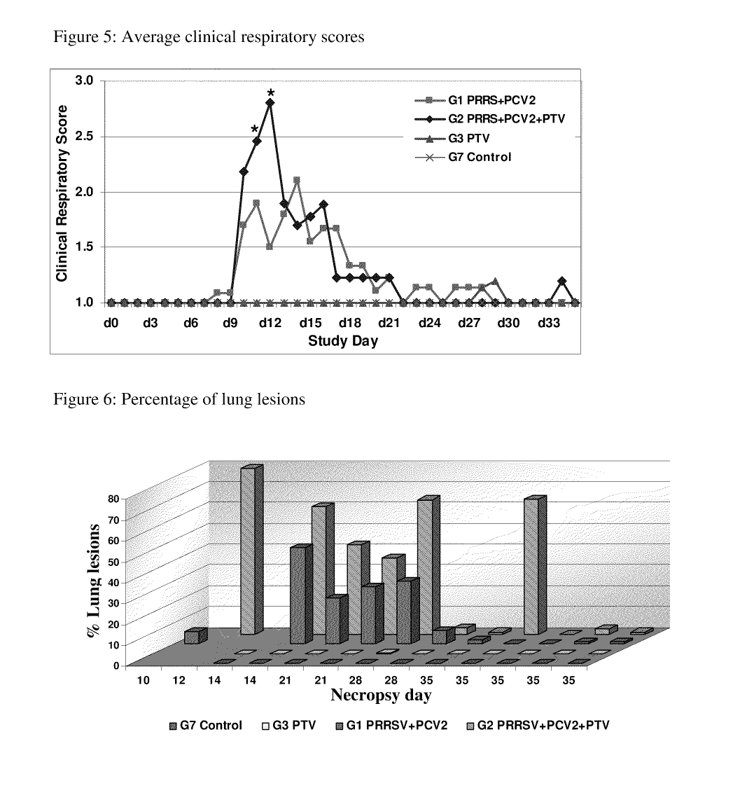

FIG. 5 shows the average clinical respiratory scores. The mean respiratory scores for each of the indicated groups were calculated at the specified study days. The error bars represent the standard error of the means. The asterisk indicates a statistical significant difference between group 1 (G1 PRRSV+PCV2) and group 2 (G2 PRRSV+PCV+PTV) at study days 11 and 12 with p<0.05.

### Rectal Temperatures

The mean rectal temperatures measured in Fahrenheit (° F.) for all groups 1 to 7 are shown in FIG. 4. As shown in FIG. 4, the average temperature expressed in degree Fahrenheit (° F.) for each group was calculated at the indicated study days. The error bars represent the standard error of the means. The asterisk indicates a statistical significant difference of the mean rectal temperatures between group 1 (PRRSV+PCV2) and group 2 (PRRSV+PCV+PTV) at study day 10 with p<0.05 based on ANOVA.

The results show that exposure to PRRSV+PCV2 induced an increase in rectal temperature >104.5° F. in the infected pigs that started to be evident in approximately 40% of pigs at day 3 post-infection. Following PTV infection there was a significant increase in rectal temperature as compared to that in the PRRSV+PCV2 exposed animals that were not infected with PTV. In contrast to groups 1 and 2, the level of rectal temperature in the other groups was not significantly different from that in the control animals.

### Mortality

Three animals were found dead on study days 10, 12, and 35. The animal that died on study day 10, in group 1 (PRRSV+PCV2) had severe CNS signs and moderate respiratory signs at days 8 & 9. This animal developed fever (>104.5° F.) at day 3 with peak temperature at 106.3° F. on day 5 and developed moderate diarrhea on day 5 and more severe diarrhea on day 7. Pig 12, which died on day 12, was in group 2 (PRRV+PCV2+PTV). This pig developed fever (>104.5° F.) on day 4, remained pyrexic until day 11, and had severe respiratory signs on days 10 and 11. The animal that died on day 35 was also in group 2, had respiratory signs on days 12-19, fever on days 3-13, and moderate CNS signs on day 34.

### Necropsy Findings

Necropsy was done on any pig that died outside of the scheduled times. Necropsy examinations were performed on two pigs from each group (except group 6) following euthanasia at the scheduled times on study days 14, 21, and 28 and on all remaining pigs at study day 35. Consistent with the clinical signs, animals in group 1 and 2 had the most extensive gross and microscopic lesions. The most consistent finding was interstitial pneumonia in the PRRSV+PCV2 exposed pigs and the gross lung lesions as noted below.

### Gross Lung Lesions

Lung lesions were more consistently observed in PRRSV+PCV2 exposed pigs at 14, 21, and 28 days post-infection. By day 35 most of the remaining animals had minimal lesions. In group 3 only one animal had minor lung lesions at study day 28 (21 days post-PTV infection) and in group 4 two animals with minimal lung lesions at study days 14 and 21 (or 7 and 14 days post-PTV infection, respectively). All other conventional animals had no visible lung lesions at time of necropsy. Among the CDCD pigs two of the PTV-infected pigs had minimal (<2%) lung lesions at day 28. The percentage of macroscopic lung lesions of each pig in groups 1 (PRRSV+PCV2), 2 (PRRSV+PCV2+PTV), and 3 (PTV) as compared to the control pigs in group 7 are shown in FIG. 6.

The results clearly show the significant contribution of PRRSV and PCV2 infection on the percentage of lung lesions compared to that of PTV infection by itself and relative to the control pigs. Although no statistically significant difference was obtained between groups 1 and 2 (p=0.1765) when comparing total scores for both groups, due to the limited number of pigs at each necropsy day and the overall variability within the groups overtime, it was clear that PRRSV+PCV2-exposed animals infected with PTV (group 2) resulted with a relatively higher percentage of gross lung lesions than pigs exposed to only PRRSV+PCV2 (group 1). The significance of PTV in the severity of the lung lesions induced by PRRSV+PCV2 requires further investigation by analyzing a greater number of pigs at earlier times after PTV infection.

FIG. 6 presents the percentage of lung lesions for each of the conventional animals from the indicated groups as determined at the necropsy date are shown as percentage of lung lesions.

### Microscopic Lesions

A summary of the number of pigs with microscopic lesions for each group is indicated in Table 5.

TABLE 5
Microscopic Lesions

| | | Number of pigs affected per group | | | | | |
|---|---|---|---|---|---|---|---|
| | Treatment | | | Lymphoid | | | |
| Group # | Group | Lung | CNS | depletion | Intestine | Heart | Spleen |
| 1 | PRRS + PCV2 | 8 | 4 | 5 | 2 | 1 | 1 |
| 2 | PRRS + PCV2 + PTV | 10 | 4 | 3 | 4 | 4 | 2 |
| 3 | PTV | 3 | 0 | 0 | 0 | 0 | 0 |
| 4 | Mix PTV | 4 | 0 | 0 | 1 | 0 | 0 |
| 5 | Reovirus | 2 | 1 | 0 | 0 | 0 | 0 |
| 6 | PTV vaccine | 1 | 0 | 0 | 0 | 0 | 0 |
| 7 | Control | 3 | 1 | 1 | 0 | 0 | 0 |

### CNS Lesions

In group 1, the pig with the most severe CNS lesions died at day 10. This pig had lesions of severe diffuse pyogranulomatous meningoencephalitis in the brain and in the spinal cord severe multifocal malacia of white matter with mild to moderate nonsuppurative vasculitis. Mild to moderate nonsuppurative meningitis was detected in the two pigs of group 1 euthanized at day 21 and in one pig at day 28. In group 2, CNS lesions were also detected in four pigs: the pig that died at day 12 had severe pyogranulomatous meningoencephalitis in the brain; one pig at day 21 had moderate patchy mixed meningitis and vasculitis with infrequent glial nodules; one of the pigs euthanized at day 28 had moderate nonsuppurative meningoencephalitis with occasional glial nodules; the other pig at day 28 had mild nonsuppurative meningitis and mild nonsuppurative meningomyelitis. In group 5 one of the pigs euthanized at day 14 had mild nonsuppurative meningoencephalitis. In group 7 one of the pigs euthanized at day 28 had mild nonsuppurative meningitis. No CNS lesions were detected in the remaining pigs.

### Lung Lesions

Consistent with what was expected for PRRSV and PCV2 exposed pigs, all animals in groups 1 and 2 that died or were euthanized before or at day 28 post-exposure had moderate to severe microscopic lung lesions mostly characterized by interstitial pneumonia. At day 35 one out of five pigs in group 1 had interstitial pneumonia and in group 2 four out of five animals had interstitial pneumonia. In group 3, lung lesions were detected in the two pigs euthanized at day 28, one with mild and the other with moderate interstitial pneumonia and in one pig at day 35 with mild interstitial pneumonia. In group 4 mild lung lesions were detected in one pig at day 21, in the two pigs euthanized at day 28 and in one pig at day 35. In group 5, one of the pigs euthanized at day 14 and one at day 28 had mild lung lesions. One of the pigs in group 6 euthanized at day 35 had mild lung lesions whereas in the control group one pig at day 28 and one at day 35 also had mild lung lesions. In the CDCD pigs four of the six PTV-exposed pigs had mild lung lesions.

### Lymphoid Tissues

The most characteristic finding was mild lymphoid depletion with some congestion in various lymphoid tissues in all pigs that were necropsied at or earlier than day 28 in group 1. In group 2, mild lymphoid depletion was detected in one pig at day 21 and the two pigs necropsied at day 28. One of the pigs in the control group that also had CNS and lung lesions at day 28 had lymphoid depletion in the spleen. In the CDCD pigs the predominant feature was reactive lymph nodes with some hemorrhage found in four of the six PTV-exposed animals and in one of the control pigs at day 28. Severe congestion in the spleen of two pigs from group 2 at study days 14 and 28.

### Intestinal Tissues

Microscopic lesions in the intestinal tract were observed in two pigs in group 1 (one with reactive Peyer's patches with apoptosis day 14 and the other with mild nonsuppurative enteritis with mild depletion of Peyer's patches at day 21), four pigs in group 2 at days 12 (mild autolysis), 14 (severe suppurative and necrotic enterocolitis), 21 (severe diffuse necrotizing and suppurative enteritis with obliteration of superficial mucosa and crypt enterocyte hyperplasia) and 35 (gut autolysis and congestion), and in one pig in group 4 with mild suppurative enteritis at day 28. No intestinal lesions were detected in any other pig.

### Cardiac Lesions

Heart lesions (multifocal suppurative vasculitis and epicarditis) were only observed in one pig of group 1 at study day 14 and in four pigs in group 2 at study days 12, 14, 21 and 28.

### Other Lesions

Serositis was detected in one pig of group 1 at study day 14.

### Average Daily Weight Gain

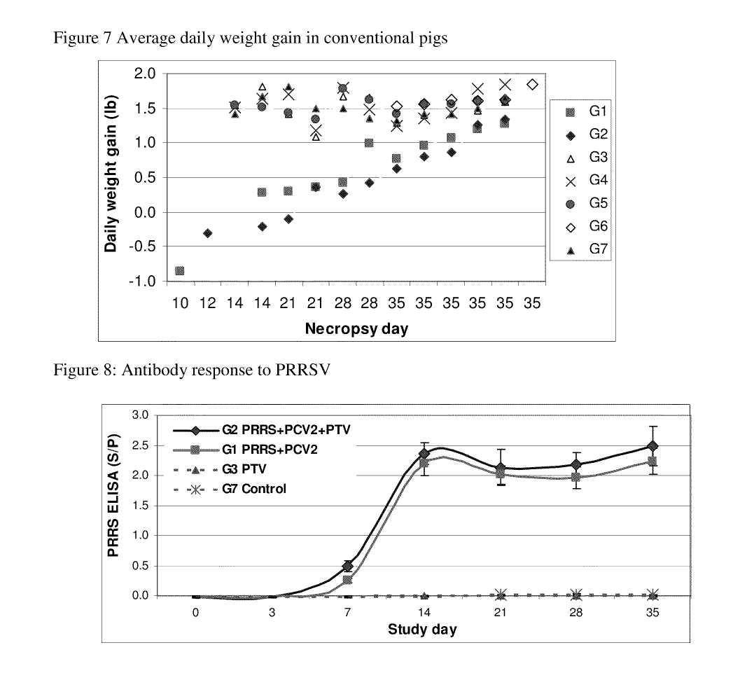

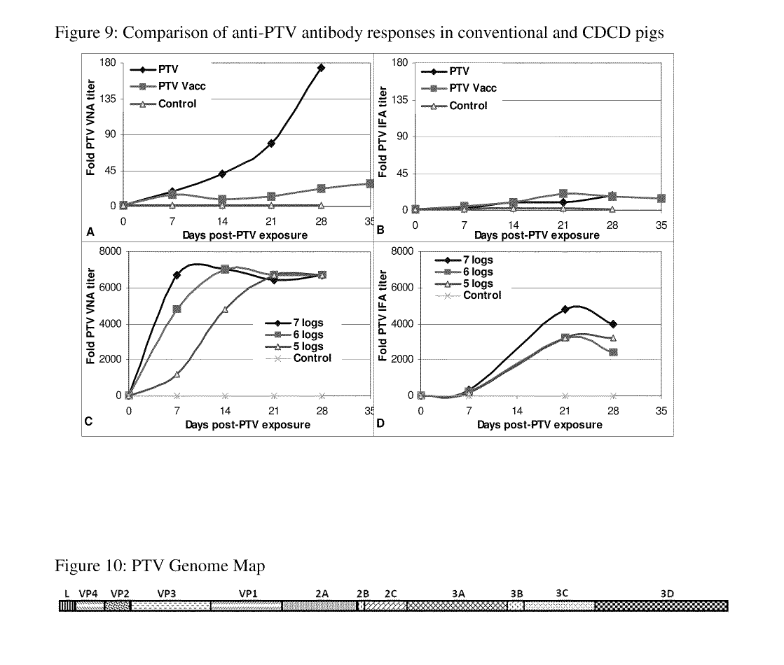

Pigs were weighed (lb) on study day 0, and at necropsy day to determine the average daily weight gain (ADWG) for each animal. The ADWG was calculated by subtracting the weight on study day 0 from the weight at necropsy day and dividing by the number of days between the measurement points. Table 6 shows the ADWG for each pig at the indicated necropsy days for all groups and the group averages and standard deviation. The data from Table 8 is represented in FIG. 7 to better visualize the difference among the treatment groups. The results show that infection with PRRSV and PCV2 had a detrimental effect on weight gain for pigs in groups 1 and 2. The negative effect on weight gain was more severe early after the infection but remained noticeable at day 35. The pigs in all other treatment groups did not significantly differ from that of control pigs. The PRRSV+PCV2-exposed pigs that were also infected with PTV tended to have a lower weight gain that the pigs not exposed to PTV. A larger number of pigs will be required to determine the significance of PTV in the severity of the weight loss induced by PRRSV+PCV2.

TABLE 6
Average Daily Weight Gain (ADWG) in conventional pigs

| | Group | | | | | | |
|---|---|---|---|---|---|---|---|
| | | G2 | | G4 | | G6 | |
| Necropsy | G1 | PRRSV + PCV2 + | G3 + | Mix | G5 | PTV | G7 |
| day | PRRSV + PCV2 | PTV | PTV | PTV | Reovirus | Vaccine | Control |
| 10 | −0.86 | | | | | | |
| 12 | | −0.30 | | | | | |
| 14 | | | 1.55 | 1.51 | 1.54 | | 1.41 |
| 14 | 0.27 | −0.21 | 1.81 | 1.64 | 1.51 | | 1.67 |
| 21 | 0.30 | −0.10 | 1.41 | 1.70 | 1.43 | | 1.80 |
| 21 | 0.35 | 0.35 | 1.09 | 1.18 | 1.33 | | 1.50 |
| 28 | 0.42 | 0.27 | 1.67 | 1.80 | 1.78 | | 1.49 |
| 28 | 0.99 | 0.43 | 1.65 | 1.49 | 1.61 | | 1.35 |
| 35 | 0.76 | 0.62 | 1.30 | 1.24 | 1.41 | 1.52 | 1.32 |
| 35 | 0.96 | 0.80 | 1.40 | 1.35 | 1.56 | 1.56 | 1.42 |
| 35 | 1.07 | 0.86 | 1.42 | 1.43 | 1.56 | 1.61 | 1.42 |
| 35 | 1.20 | 1.26 | 1.47 | 1.78 | 1.61 | 1.61 | 1.49 |
| 35 | 1.27 | 1.34 | 1.59 | 1.84 | 1.61 | 1.63 | 1.65 |
| 35 | | | | | | 1.84 | |
| Average | 0.61 | 0.48 | 1.49 | 1.54 | 1.54 | 1.63 | 1.50 |
| STDEV | 0.61 | 0.56 | 0.20 | 0.23 | 0.12 | 0.11 | 0.15 |

### Virus Isolation

Recovery of viruses from the infected pigs were confirmed based on CPE and staining with virus-specific antibodies for PRRSV in AK-MA104 cells, and for PTV in PKWRL and ST cells. Isolation of PCV2 was based on specific staining in VIDO-R1 cells. Table 7 summarizes the results of virus isolation.

TABLE 7
Summary of Virus Isolation from conventional pigs.

| | | Number of positive | | |
|---|---|---|---|---|
| | Treatment | pigs per group | | |
| Group # | Group | PRRSV | PCV2 | PTV |
| 1 | PRRS + PCV2 | 11 | 3 | 1 |
| 2 | PRRS + PCV2 + PTV | 11 | 5 | 6 |
| 3 | PTV | 0 | 0 | 2 |
| 4 | Mix PTV | 0 | 0 | 0 |
| 5 | Reovirus | 0 | 0 | 0 |
| 6 | Killed PTV vaccine | 0 | 0 | 0 |
| 7 | Control | 0 | 0 | 0 |

PRRSV and PTV based on CPE and IFA. PCV2 based on IFA.

### PRRSV Isolation

Viremia was confirmed in all PRRSV-infected pigs in groups 1 and 2 at days 3 and 7 post-infection. PRRSV was also isolated from serum at day 14 in 70-80% of the infected pigs and from various tissues from three pigs in group 1 and from one pig in group 2. Interestingly, the pig that died at day 10 in group 1 was positive for PRRSV in the brain. The second pig from group 1 was PRRSV positive in the tonsil at day 21 and the third pig in group 1 was PRRSV positive in the lung, tonsil and lymph nodes at day 28. In group 2, one pig was positive in the lung at day 28. No PRRSV was confirmed in the tissues of any other pig in the groups 1 and 2. Serum and tissue samples from the conventional pigs in all other groups were negative for PRRSV isolation.

### PCV2 Isolation

PCV2 was isolated from brain, spinal cord, tonsil, lymph nodes, and spleen from the two pigs euthanized at day 21 and from one of the pigs euthanized at day 28 in group 1. In group 2, PCV2 was also isolated from various tissues of five pigs, one at day 21, one at day 28 and three at day 35. No PCV2 was isolated from any other pig based on IFA staining of inoculated VIDO-R1 cells with anti-PCV2-specific antibody.

### PTV Isolation

Isolation of PTV from the PTV-infected pigs was confirmed in the serum of six pigs at day 14 (7 days post-PTV infection) and from the lung of one pig euthanized at day 35 in group 2. In group 3, PTV was only isolated from the tonsil of two pigs, one euthanized at day 21 and one at day 35. Unexpectedly, PTV was also isolated from one pig of group 1, detected in serum at day 14 and in brain at day 35. PTV was not isolated from any conventional pig in groups 4, 5, 6 or 7. Among the CDCD pigs infected with PTV, the virus could only be recovered from the serum of one pig at day 7 post-infection.

### Serology

### Pre-Screening for PRRSV, PCV2 and PTV Antibodies

To assess the serological status of the conventional pigs, serum samples from three piglets per litter representing twenty litters were analyzed and the data used to select the pigs to be used in the study. Based on the results, one litter that had anti-PRRSV antibodies was eliminated. However, for PTV and PCV2, all piglets had variable levels of maternal antibodies, and most of them had high anti-PCV2 antibody titers. To minimize the effect of maternal immunity, piglets were re-evaluated for anti-PCV2 and anti-PTV antibodies at study day −14 and were acclimated for two more weeks. Despite some decay of maternal immunity only 28% of piglets were negative for anti-PCV2 antibodies, approximately 53% had high antibody titers and about 19% with relatively low anti-PCV2 antibody titers. The anti-PTV antibodies were relatively low however, at study day 0a considerable number (70%) of pigs had anti-PTV antibody titers ≧1:100 whereas approximately 63% of pigs had anti-PCV2 antibody titers of >1:200 and all pigs were negative for anti-PRRSV antibodies.

### PRRSV Serology

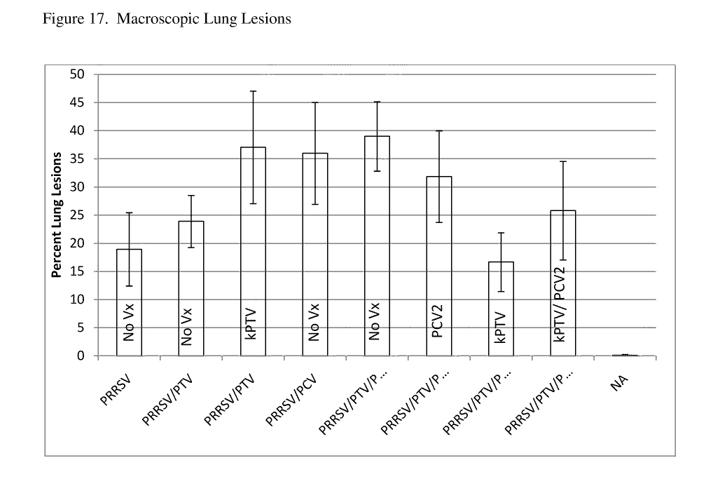

Seroconversion was demonstrated in all pigs that were infected with PRRSV in groups 1 and 2. No other pig, conventional or CDCD, developed antibodies to PRRSV during the duration of the study. FIG. 8 shows the serological response for groups 1 (PRRSV+PCV2), 2 (PRRSV+PSV2+PTV) and 3 (PTV) as compared to the control pigs. FIG. 17 illustrates the antibody response to PRRSV. No significant difference in the overall serological response to PRRSV observed between groups 1 and 2 (p>0.05).

### PCV2 Serology

The level of maternal anti-PCV2 antibodies in the conventional pigs at the initiation of the study confounded the serological response to PCV2. To get a better assessment of the PCV2 antibody response, the fold increase in antibody titers at various times after infection relative to day 0 was calculated and the relevant data for pigs in the groups 1, 2, and 3 as compared to the control animals are shown in Table 8. Based on a >2-fold increase in antibody titers relative to study day 0, seroconversion was detected in 4 pigs from group 1 and in 8 pigs from group 2 at day 14. One pig in group 1 remained with relatively high titers at day 35 and one pig in group 2 with high titer at day 28. In contrast, all other pigs in these two groups had a rapid decline in anti-PCV2 antibody titers with 3 out 6 pigs in group 1 and all 6 pigs in group 2 having undetectable anti-PCV2 antibodies by day 35. Unexpectedly, an increase in anti-PCV2 antibody titers was also observed in 3 pigs from group 6, immunized with killed PTV virus, with antibody titers remaining relative high at day 35 in two of the pigs. One of the control pigs also had an increase in antibody titer at day 28 with rapid decline at day 35. No increase but a gradual decline in antibody titers to PCV2 were detected in the conventional pigs from groups 3, 4, and 5.

TABLE 8
Fold Increase in Anti-PCV2 Antibody Titers for Groups 1, 2, 6, and 7.

| | | Necr | Day post-PCV2 infection | | | | Pig | | Necr | Day post-PCV2 infection | | | |
|---|---|---|---|---|---|---|---|---|---|---|---|---|---|
| Pig ID | Group # | day | D0 | D14 | D28 | D35 | ID | Group # | day | D0 | D14 | D28 | D35 |
| 202 | 1 | d10 | 1.00 | NS | NS | NS | 112 | 2 | d12 | 1.00 | NS | NS | NS |
| 137 | 1 | d14 | 1.00 | 32.00 | NS | NS | 210 | 2 | d14 | 1.00 | 64.00 | NS | NS |
| 146 | 1 | d21 | 1.00 | 32.00 | NS | NS | 154 | 2 | d21 | 1.00 | 26.67 | NS | NS |
| 201 | 1 | d21 | 1.00 | 1.00 | NS | NS | 178 | 2 | d21 | 1.00 | 0.50 | NS | NS |
| 166 | 1 | d28 | 1.00 | 2.00 | 0.13 | NS | 177 | 2 | d28 | 1.00 | 130.00 | 32.00 | NS |
| 212 | 1 | d28 | 1.00 | 1.00 | 1.00 | NS | 195 | 2 | d28 | 1.00 | 4.00 | 0.06 | NS |
| 138 | 1 | d35 | 1.00 | 2.67 | 1.00 | 8.00 | 134 | 2 | d35 | 1.00 | 16.00 | 0.17 | 0.13 |
| 161 | 1 | d35 | 1.00 | 2.00 | 0.04 | 0.03 | 159 | 2 | d35 | 1.00 | 16.25 | 0.06 | 0.06 |
| 175 | 1 | d35 | 1.00 | 0.25 | 0.04 | 0.02 | 169 | 2 | d35 | 1.00 | 4.06 | 0.06 | 0.02 |
| 189 | 1 | d35 | 1.00 | 4.00 | 0.06 | 0.03 | 188 | 2 | d35 | 1.00 | 2.00 | 0.50 | 0.25 |
| 196 | 1 | d35 | 1.00 | 0.13 | 0.13 | 0.06 | 191 | 2 | d35 | 1.00 | 8.13 | 0.06 | 0.03 |
| 111 | 6 | d35 | 1.00 | 1.00 | 64.00 | 8.00 | 136 | 7 | d14 | 1.00 | 0.67 | NS | NS |
| 163 | 6 | d35 | 1.00 | 1.00 | 1.00 | 0.50 | 179 | 7 | d14 | 1.00 | 1.00 | NS | NS |
| 176 | 6 | d35 | 1.00 | 0.50 | 0.25 | 0.25 | 153 | 7 | d21 | 1.00 | 2.00 | NS | NS |
| 198 | 6 | d35 | 1.00 | 0.50 | 2.00 | 0.50 | 185 | 7 | d21 | 1.00 | 0.50 | NS | NS |
| 199 | 6 | d35 | 1.00 | 8.00 | 130.00 | 64.00 | 168 | 7 | d28 | 1.00 | 0.50 | 0.10 | NS |
| 209 | 6 | d35 | 1.00 | 1.00 | 64.00 | 32.00 | 207 | 7 | d28 | 1.00 | 0.50 | 0.33 | NS |
| | | | | | | | 129 | 7 | d35 | 1.00 | 1.00 | 1.00 | 0.50 |
| | | | | | | | 148 | 7 | d35 | 1.00 | 1.52 | 1.52 | 1.52 |
| | | | | | | | 171 | 7 | d35 | 1.00 | 0.75 | 0.50 | 0.19 |
| | | | | | | | 187 | 7 | d35 | 1.00 | 0.50 | 8.00 | 0.50 |
| | | | | | | | 197 | 7 | d35 | 1.00 | 0.67 | 0.50 | 0.13 |

NS = no sample available.
Numbers represent ratio of antibody titers at the indicated study day relative to day 0.
Bold numbers indicate sero-conversion with >2 fold increase in antibody titer relative to day 0

### PTV Serology

The serological response to PTV was analyzed by virus neutralization antibody (VNA) test in all pigs of the study. In addition, indirect immuno-fluorescence antibody (IFA) assay was performed for pigs in groups 3, 6, and 7. Given that a large percentage (˜70%) of pigs had anti-PTV antibody titers at the day of PTV exposure, the fold increase of antibody titers relative to time of PTV infection (study day 7 for groups 3 and 4) or killed PTV vaccination (study day 0 for group 6) was calculated. Based on a fold increase >2, seroconversion was detected in 9%, 45%, 73%, 55%, 36%, 100%, and 36% of pigs in groups 1 to 7, respectively. However, the levels of antibody response in the positive pigs from groups 1, 5 and 7 were minimal. In contrast, a significant increase (p<0.05) in antibody titers was observed in the pigs of group 3 that were infected with the single PTVp6137A-1 isolate and to a lesser extent in the PTV-exposed pigs from groups 2, 4 and 6.

The average fold increase in anti-PTV antibody titers for all groups shown in table 9, clearly demonstrate the significant (p<0.05 based on Kruskal-Wallis/Wilcoxon Two Sample Test) serological response in the PTV infected and vaccinated pigs compared to the non-PTV infected and control pigs.

TABLE 9
Average Fold Increase in Anti-PTV Neutralizing Antibody
Titers in Conventional Pigs

| Fold | | | | | | | |
|---|---|---|---|---|---|---|---|
| increase | | | | | | | |
| VNA | | Days post-experimental PTV exposure | | | | | |
| titer | Group | 0 | 7 | 14 | 21 | 28 | 35 |
| Average | G1 | 1 | 0.84 | 0.94 | 2.91 | 2.90 | NA |
| | G2 | 1 | 1.48 | 1.19 | 4.45 | 12.25 | NA |
| | G3 | 1 | 18.09 | 40.56 | 79.43 | 174.40 | NA |
| | G4 | 1 | 1.36 | 1.78 | 6.71 | 11.20 | NA |
| | G5 | 1 | 1.14 | 1.67 | 4.07 | 1.30 | NA |
| | G6 | 1 | 14.33 | 8.83 | 12.67 | 22.00 | 28.00 |
| | G7 | 1 | 1.55 | 1.56 | 1.71 | 0.80 | NA |
| Standard | G1 | 0 | 0.22 | 0.40 | 1.89 | 1.31 | NA |
| Error | G2 | 0 | 0.75 | 0.39 | 2.02 | 6.79 | NA |
| | G3 | 0 | 12.49 | 27.22 | 22.28 | 47.41 | NA |
| | G4 | 0 | 0.23 | 0.81 | 2.44 | 5.28 | NA |
| | G5 | 0 | 0.18 | 0.46 | 2.06 | 0.30 | NA |
| | G6 | 0 | 9.97 | 4.81 | 4.55 | 9.55 | 11.59 |
| | G7 | 0 | 0.38 | 0.37 | 0.42 | 0.12 | NA |

Fold increase calculated by dividing the arithmetic titer at the indicted day by the titer obtained at day of experimental PTV exposure (study day 7 for groups 1, 2, 3, 4, 5, and 7 and study day 0 for group 6).
Fold increase in antibody titers >2 are in bold.
NA = not tested at day 35 post-experimental PTV exposure.

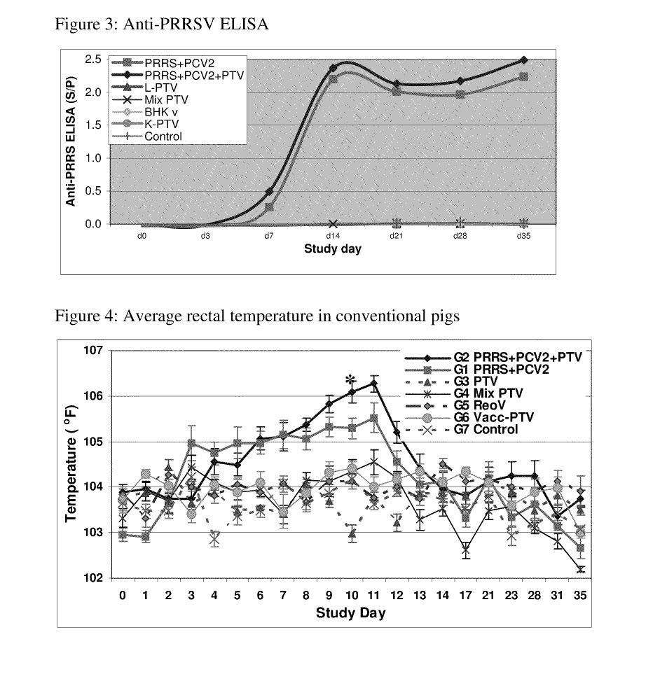

From the data shown in FIGS. 3 and 8 it is also evident the delayed and significantly (p<0.05) reduced level of antibody response in the PTV infected pigs previously exposed to PRRSV and PCV2 (group 2) compared to the pigs that were only infected with the corresponding single PTV isolate (group 3). In contrast, the vaccinated pigs with the killed PTV (group 6) had a rapid antibody response at day 7 after first PTV exposure at similar level to that in pigs of group 3, and a slight increase in antibody response upon the second vaccine dose.

FIG. 8 illustrates serological response to PTV. The ratio of the antibody titer at the indicated time post-PTV exposure to the titer at time of PTV exposure was calculated for each animal and the average of the fold increase in anti-PTV neutralizing antibody titers is shown for conventional pigs in group 1 (G1 PRRSV+PCV2), group 2 (G2 PRRSV+PCV2+PTV), group 3 (G3 PTV), group 6 (G6 killed PTV vaccine) and group 7 (G7 control). Arrows indicate time of PTV exposure: day 0 corresponds to study day 7 for PTV infected pigs and study 0 for killed PTV vaccinated pigs (PTV Vacc). The vaccinated pigs received a second dose at study day 14.

The pigs in group 4 exposed to a mixture of PTV isolates different from the isolate used in groups 2, 3 and 6 also had a delayed and lower anti-PTV neutralizing antibody response than those in group 3 that were infected with the single PTVp6137A-1 isolate.

### Conclusions

The major goal of the study was to evaluate the pathogenicity of the viruses isolated from swine disease outbreak investigation, specifically to determine whether the severe CNS clinical disease could be reproduced in conventional pigs upon exposure to the PTV isolate in the presence or absence of PRRSV and PCV2 infection. In addition, the study also evaluated the killed PTV vaccine concept by demonstrating the development of PTV-specific antibodies in the immunized pigs and the pathogenesis of the unidentified reovirus.

Neurological signs were observed in 5 out of 11 pigs infected with PRRSV and PCV2 in group 1 and in one pig in group 2. The neurological signs were, in general, mild except for one pig in group 1 that developed severe CNS clinical signs, died early after infection and showed severe microscopic CNS lesions. In addition, microscopic CNS lesions were also observed in approximately 36% (4 out of 11) of the PRRSV+PCV2 infected pigs. Given that the same number of pigs in group 1 and group 2 had microscopic lesion in the CNS, and more pigs in group 1 than in group 2 developed CNS signs, a role of PTV in the CNS disease was not demonstrated in this study.

One important factor that may have contributed to the reduced percentage of affected pigs and mild CNS disease could be the presence of maternal antibodies to PTV and PCV2 in the pigs used in this study, despite efforts to reduce their significance by allowing pigs acclimate longer and using antibody titer levels to PCV2 and to PTV as selection criteria. Even though antibody decay was demonstrated, the level of antibodies may have been sufficient to prevent the spread of PTV and/or PCV2 to the CNS, and therefore, prohibiting the development of CNS signs in the infected pigs. In support of the potential role of maternal immunity in prohibiting the CNS disease, is the observation that PCV2 recovery from the brain of infected pigs by virus isolation was from pigs that had the lowest or undetectable anti-PCV2 antibodies at the initiation of the study and also had microscopic CNS lesions. Therefore, a more comprehensive analysis of the role of PCV2 and PTV in the CNS disease requires the use of animals lacking maternal immunity.

The study also showed that PTV infection either as single isolate or as a mix of PTV isolates did not induce obvious clinical signs in the conventional pigs. However, PTV infection induced mild lung and lymph node lesions. The preliminary assessment of the pathogenicity of PTV in the small number of CDCD pigs that lacked maternal immunity to PTV, confirmed that the PTV by itself and under the conditions evaluated does not induce CNS disease or other apparent clinical signs. However, the single PTV virus infection in the conventional and CDCD pigs induced microscopic lesions in the lung and lymph nodes and a rapid and robust serological response.

The reduction of anti-PTV antibodies in the group previously infected with PRRSV-PCV2 compared to animals infected with PTV alone may explain both the increase in intestinal lesions and the higher recovery of PTV from the infected pigs and also support the hypothesis that PRRSV and PCV2 may be predisposing factors for PTV-induced disease.

Despite the lack of direct PTV induced disease, this study demonstrated that PTV exposure with live virus or with killed PTV vaccine induced a significantly high antibody response relative to the control pigs (p<0.05). The feasibility of using a killed virus for induction of PTV-specific antibody responses was supported by the level of antibodies in the vaccinated pigs that reached similar levels to those detected in the live virus infected pigs when measured by indirect fluorescent antibody test. However, infection with live virus induced a more robust serum neutralizing response. Further studies are necessary to determine the level of neutralizing antibodies required for protection when a suitable challenge model is established. Furthermore, the low level of antibody response detected in the animals infected with the mix of PTV isolates in group 4, which were different from the PTVp6137A-1 isolate used as antigen in the serological assay, may reflect an antigenic difference among the isolates that need to be demonstrated in future analyses.

Most importantly, this study, provided support for an important role of PTV in the respiratory disease complex induced by PRRSV and PCV2. This was evidenced by a significant increase in the magnitude of respiratory clinical signs and rectal temperature (p<0.05), by an increase in level of gross lung lesions, by an increase in the severity of microscopic lung lesions, especially at later time of infection, and by an increase in the number pigs with microscopic lesions in the intestinal tract, spleen and heart upon PTV exposure of PRRSV+PCV2-infected pigs compared to the results obtained in the pigs that were not exposed to PTV. In addition, it was apparent that PRRSV+PCV2-infected pigs exposed to PTV had a lower daily weight gain than pigs not exposed to PTV. The more dramatic effect of PTV exposure in the PRRSV+PCV2 infected pigs were observed between study days 10-28. Recognizing that only two pigs were euthanized at each time point during this study, it is important to confirm the significance of PTV infection in the complex respiratory disease model with a larger number of pigs per observation at earlier times post exposure.

The degree at which the PRRSV- and PCV2-infected pigs developed clinical respiratory signs, fever, lung lesions and diminished performance was markedly increased by PTV infection in affected pigs. These data provide relevant evidence of an important role of PTV in the respiratory disease induced by PRRSV in the presence of PCV2.

In addition, the significant lower anti-PTV antibody responses in the group previously infected with PRRSV-PCV2 compared to animals infected with PTV alone may explain both the increase in the number of pigs with microscopic lesions in the intestinal tract, spleen and heart and the higher recovery of PTV from the PRRSV+PCV2-infected pigs than in the PTV-only infected pigs. These findings support the hypothesis that PRRSV and or PCV2 may be predisposing factors for PTV-induced disease that may be more apparent in the absence of maternal immunity.

Furthermore, this study demonstrated that immunization with a killed PTV virus induces a neutralizing antibody response and this provides additional support of a PTV vaccine concept.

### Example 2

### Objective

(1) To determine whether passively acquired maternal antibodies or virus dose effect PTV pathogenicity in colostrums-deprived-cesarean-derived (CDCD) animals.

### Materials and Methods

### Experimental Design

The study consisted of two groups (group 8 and 9) of CDCD pigs which were inoculated with PTV or shaminoculated with MEM (minimual essential media; negative controls). Table 10 outlines the number of animals per group and the viral strains with which they were inoculated. Experimental group 8 consisted of six animals that were inoculated with PTV at three different doses with two pigs per dose. Group 9 consisted of two negative control pigs that were sham inoculated with media Animals were observed daily following inoculation for the presence of abnormal clinical signs. All animals were bled on study days 0, 4, 7, 14, 21, 28. Animals were humanely euthanized on study day 28.

TABLE 10
Outline of treatment group inoculum and dose information.

| Group | n | Viral inoculum | Viral titer |
|---|---|---|---|
| 8 | 2 | PTV sw022208-6137A-1 | 7 logs TCID₅₀/ml |
| | 2 | PTV sw022208-6137A-1 | 6 logs TCID₅₀/ml |
| | 2 | PTV sw022208-6137A-1 | 5 logs TCID₅₀/ml |
| 9 | 2 | MEM | Not applicable |

### Sample and Data Collection

### Whole Blood Collection

Blood was collected from conventional pigs on days −18, 0, 3 or 4, 7, 14, 21, 28, and 35 and separate aliquots for serology and virus isolation prepared.

### Clinical Observations

Animals were observed daily for clinical signs from day 0 to day 35. Special attention was placed to determine development of neurological abnormalities such as lack of coordination, tremors, sternal and/or lateral recumbency, convulsions, paralysis or inability to stand or walk, etc. in the infected pigs and or reactivity in the vaccinated pigs. Other clinical signs including respiratory signs and diarrhea were also noted and recorded. The clinical condition of these animals was evaluated based on a numerical index reflecting the severity of illness. Scores for each of the individual observations ranged from 1 to 3 with 1 assigned for a normal condition, 2 for mild condition and score of 3 for a severe condition. The total score consisted of the sum of the daily observations for each abnormal observation. A dead animal as a result of the infection was given a total score of 4 for each condition observed.

### Rectal Temperatures

Measurements were recorded daily from study day 0 to 14 and twice a week thereafter.

### Necropsy

The brain, spinal cord, tonsil, thymus, lung, heart, spleen, lymph nodes, spleen, liver, kidney and intestinal organs of the study animals were evaluated for evidence of gross lesions as compared to sham-infected control pigs. The percentage of lung lesions was determined and scored for each pig at necropsy and lesions in any other tissue were also recorded if present. Samples from brain, spinal cord, tonsil, thymus, lung, heart, spleen, lymph nodes, liver and intestinal tract were collected in 10% buffered formalin and submitted to ISU-VDL for histopathology analysis to determine microscopic lesions. Another set of samples that included brain, spinal cord, tonsil, lung, lymph nodes, and spleen were collected for virological evaluation. Tissue samples for virological analysis were collected and shipped in dry ice and/or stored at −70° C. until processing. Tissue homogenates were prepared in basal media as 5-10% suspension, clarified by centrifugation and filtered through 0.22 μm filters. Aliquots were stored at −70° C. until ready for analysis.

### Serological Evaluation

Serum samples were tested for antibodies to PRRSV, PCV2 and PTV. Antibodies to PRRSV were measured by IDEXX ELISA and reported as S/P ratios by the Boehringer Ingelheim Vetmedica, Inc. Health Management Center Diagnostic lab in Ames, Iowa. Antibodies to PCV2 were measured by indirect fluorescence antibody (IFA) test and antibody titers were reported as the mean of the reciprocal of last dilution with specific fluorescence. Antibodies to PTV were measured by virus neutralization assay (VNA) and a subset of samples was also tested by IFA. For PTV serology, the PTV-p6137A-1 isolate as used as antigen for the assay results reported in this study. The VNA was performed with heat-inactivated serum. Anti-PTV antibody titers were reported as the mean of the reciprocal of last dilution that neutralized virus-induced CPE for VNA or that showed specific florescence for IFA. Antibody responses in the animals exposed to the unidentified reovirus were also analyzed by IFA.

### Viral Assays

Serum and tissue homogenates were analyzed for virus isolation to confirm the recovery of infectious PRRSV, PCV2, PTV and the unidentified reovirus. Virus isolation was performed by inoculating two-day old monolayers prepared in 96-well plates of the following cell lines: AKMA104, VIDO-R1, PK/WRL, PK2a, ST BHK21 using approximately 20 ul sample/well and 4 wells per sample. Virus Isolation was confirmed based on CPE and immuno-fluorescence staining of pass 3 cultures with available virus-specific antibodies. PRRSV isolation was confirmed based on CPE in AK-MA1904 cells and staining with the SR-30 monoclonal anti-PRRSV antibody. PCV2 isolation was confirmed based on staining of pass 3 VIDO-R1 with anti-PCV2 ORF2 monoclonal antibody. PTV isolation was confirmed based on CPE in PKWRL, PK2a or ST cells and staining with a polyclonal swine anti-PTV/PEV antibody. The reovirus isolation was confirmed by PCR of pass 3 BHK21 cultures.

### Results

### Clinical Observations

Animals were observed daily for the duration of the study and monitored for the development of clinical signs that included neurological signs, mortality, respiratory disease, and diarrhea. No significant neurological signs were observed in animals inoculated with PTV alone. None of the CDCD pigs developed diarrhea during the duration of the study. No clinical respiratory signs were noted in any of the CDCD animals during the duration of the study. The rectal temperature in the CDCD pigs remained below 104.5° F. during the 28 day study period.

### Gross Lung Lesions

Two of the PTV-infected pigs had minimal (<2%) lung lesions at day 28.

### Microscopic Lesions

### Lung Lesions

Four of the six PTV-exposed pigs had mild lung lesions.

### Lymphoid Tissues

The predominant feature was reactive lymph nodes with some hemorrhage found in four of the six PTV-exposed animals and in one of the control pigs at day 28.

### Other Tissues

No intestinal, cardiac, or CNS lesions were noted in any of the pigs.

### Virus Isolation

PTV was only recovered from the serum of one pig at day 7 post-infection.

### Serology

### Pre-Screening for PRRSV, PCV2 and PTV Antibodies

As expected, CDCD pigs were negative for antibodies to PRRSV, PCV2 and PTV at study day 0.

### PTV Serology

The CDCD pigs had undetectable anti-PTV neutralizing and IFA antibodies at the time of infection and rapidly developed antibodies with titer levels that were dependent on virus dose at day 7 post-infection. The anti-PTV neutralizing antibody titers rapidly increased to the maximum serum dilution evaluated (1:6400) and remained high for the duration of the experiment. In contrast the IFA titers tended to decrease by day 28. The level of anti-PTV antibody response measured by IFA and VNA tests in animals in Example 1 compared to the response in CDCD pigs (Example 2) is shown in FIG. 19. The results show that the serological response to PTV when measured by IFA developed at a slower rate and at lower level that the VNA response in the infected pigs. However, in the vaccinated animals, the IFA response reached similar levels to that of the VNA response albeit at slower rate.

### Conclusions

Singular PTV virus infection in animals without maternal immunity to PTV induced a rapid and robust serological response. However, singular PTV infection of CDCD animals did not induce CNS disease or other apparent clinical signs.

### Example 3

### Objectives

(1) To determine whether concurrent exposure to porcine respiratory and reproductive syndrome virus (PRRSV) and porcine circovirus type 2 (PCV2) prior to exposure to PTV enhance CNS signs in cesarean-derived colostrum-deprived (CDCD) pigs.

(3) To evaluate the role of PTV in respiratory disease induced by PRRSV or by PRRSV and PCV2 co-infection in CDCD pigs.

### Materials and Methods

### Experimental Design

The study consisted of seven experimental groups and one control group (Table 11). Each group contained 14 or 15 pigs depending on health status at day 0. On day 0, all animals were inoculated with the indicated treatment (Table 11). Throughout the duration of the study, animals were observed daily for abnormal clinical signs. All animals were weighed on days 0, 7, 14, 21, and 28. Blood was collected from all animals on days 0, 3, 7, 14, 17, 21, and 28. The study was terminated on day 28.

TABLE 11
Treatment groups

| Group | | | Treatment | Treatment |
|---|---|---|---|---|
| No. | n | Group name | Day 0* | Day 7** |
| 1 | 14 | PRRSV | PRRSV | MEM |
| 2 | 14 | PRRSV + PTV | PRRSV | PTV |
| 3 | 14 | PCV2 | PCV2 | MEM |
| 4 | 14 | PCV2 + PTV | PCV2 | PTV |
| 5 | 15 | PRRSV-PCV2 | PRRSV + PCV2 | MEM |
| 6 | 14 | PRRSV-PCV2 + | PRRSV + PCV2 | PTV |
| | | PTV | | |
| 7 | 15 | PTV | MEM | PTV |
| 8 | 14 | Control | MEM | MEM |

*The treatment on day 0 was given intranasally; total volume of the inoculum was 2 ml. PRRSV = Porcine reproductive and respiratory syndrome virus, BI internal reference strain PRRSV-972-1 propagated in AKMA104 cells to limiting dilution (LD) pass 4 and diluted in MEM with 4% FBS to a titer of 4.5 logs TCID₅₀/ml; PCV2 = porcine circovirus type 2, BI internal reference strain PCV2-194-8 propagated in VIDO-R1 cells to pass 2 and diluted in MEM with 4% FBS to a titer of 4 logs TCID₅₀/ml; PTV = porcine teschovirus, BI internal reference strain PTV-6137A-1 propagated in PK-WRL cells to LD pass 4 and diluted in MEM with 4% FBS to a titer of 6.5 logs TCID₅₀/ml; and MEM = minimal essential media (negative control incolumn).
**The treatment on day 7 was given intravenously; total volume of the inoculum was 2 ml.

### Animal and Housing Information

Colostrum-deprived caesarian-derived (CDCD) pigs were received at 7 weeks of age. After a health examination confirmed that the animals were in good health condition, animals were randomly allocated to treatment groups. Each experimental group was allocated in a separate pen within a room with two groups with the same first treatment at day 0 per room (groups 1 and 2, 3 and 4, 5 and 6, 7 and 8). Pens consisted of solid sided pens preventing any direct contact between pens. To minimize potential for cross contamination between groups within the same room, pens from the two treatment groups were curtained off from each other, hepa-filtered, and separate supplies and boots, tyveks, gloves were used for each treatment group. In addition, treatment groups within the same room were observed and handled in an order to minimize potential for cross contamination (example—group 1 pens then group 2 pens).

### Sample or Data Collection:

### Whole Blood Collection

Whole blood for sera production was collected on days 0, 3, 7, 10, 14, 21, and 28. Approximately 10-14 ml whole blood was collected from each pig, except at the day of euthanasia for pigs in groups 7 and 8 (PTV-infected and control groups, respectively) from which at least 50 ml of blood was collected to obtain reference serum. Aliquots of serum samples were prepared for virus isolation, serological evaluation and to submit to ISU-VDL or PCR analysis if necessary. Aliquots from serum samples collected at euthanasia were kept frozen as reference sera for further studies based on antibody titers.

### Weights

Animals were weighed at study day 0 prior to the treatment and at necropsy date, to monitor the weight gain of all animals.

### Clinical Observations

Animals were monitored daily for clinical signs from day 0 to day 28. Special attention was placed on the development of neurological abnormalities, respiratory disease, diarrhea, fever, and body condition (effect on weight gain). Neurological signs may include depression, uncoordination, tremors, sternal and or lateral recumbency, convulsions, partial paralysis or inability to stand or walk. Manifestation of fever (which will be determined based on rectal temperature records) was recorded daily from study day 0 to 14 and twice a week thereafter. The clinical condition of these animals was evaluated based on a numerical index that reflects the severity of illness. Scores for each of the individual observations ranged from 0 to 3 where 0 indicates a normal condition, 1 indicates a mild condition, 2 indicates a moderate condition and 3 indicates a severe condition.

### Necropsy

In the absence of clinical signs, euthanasia was performed in randomly selected pigs according to the following schedule: at study day 14 euthanasia was performed in eight pigs from group 1, 2, 3, 4, 5, 6 and 8 and in five pigs from group 7; at study days 21 and 28 euthanasia was performed in three pigs from groups 1, 2, 3, 4, 5, 6 and 8 and in five pigs from group 7. If clinical signs were present at the scheduled euthanasia dates, pigs showing higher clinical scores were selected over pigs with low clinical signs for necropsy. Pigs developing severe clinical signs were euthanized at the peak of disease manifestation if different from the scheduled necropsy dates. In this case and if the actual number of pigs in the study were reduced for other reasons, the number of pigs euthanized at the scheduled days was adjusted accordingly. Euthanasia was performed by sedation, followed by electrocution and rapid exsanguination. Necropsy was performed in all euthanized pigs for a detailed analysis of macroscopic lesions and tissue sample collection. The brain, spinal cord, tonsil, thymus, lung, heart, spleen, lymph nodes, spleen, liver, kidney and intestinal organs of the study animals were evaluated and scored at necropsy for evidence of gross lesions and compared to sham-infected control pigs. An important parameter to evaluate the severity of the respiratory disease is the percentage of lung lesions. The total lung score was determined based on the areas affected and recorded in the necropsy report form. Description of lesions observed in all other affected tissues was also recorded. To decrease the possibility of cross-contamination with intestinal contents, the intestinal tract was examined and sampled last if deemed necessary based on clinical signs and gross lesions. Tissue samples were collected for virus isolation, histopathology and bacteriological analyses as indicated in the corresponding assay section. For virus isolation, samples were collected in sterile bags, each bag containing an individual tissue and immediately frozen (store −70 C and ship in dry ice). For histopathology analysis, tissue sections were collected in 10% buffered formalin and stored at room temperature until all samples are collected and were submitted for analysis at the end of the study. If clinical observations and gross lesions were indicative of bacterial infection, a third set of samples were collected and, immediately after necropsy, submitted for bacteriological analysis.

### Serological Evaluation

Serum samples collected throughout the study were analyzed for the presence of antibodies against PRRSV, PCV2, and PTV. Antibodies to PRRSV will be demonstrated by commercial IDEXX ELISA. Antibodies to PCV2 were evaluated by indirect immuno-fluorescence assay (IFA). Antibodies to PTV were analyzed by serum neutralization (SN) test and IFA.

### Viral Assays

For a quantitative assessment of PRRSV and PCV2 viremia levels, corresponding viral nucleic acids were measured in serum samples by real time PCR. Serum and tissue samples was analyzed for virus isolation to confirm the recovery of infectious PRRSV, PCV2 and PTV using AKMA104, VIDO-R1 and PK/WRL cells, respectively. Tissue samples were prepared for virus isolation by homogenization and suspended in basal media and antibiotics to approximately 5%-10% tissue suspension, clarified by centrifugation and filtered through a 0.02 um filters. Aliquots of serum and tissue homogenate suspensions were be stored at −70 C until analysis. Single use aliquots were used for the virus isolation. Tissue homogenate samples analyzed for virus isolation included a pool of various sections of the brain, a pool of various sections of the spinal cord (including brain stem, cervical, thoracic, lumbar and sacral region), tonsil, lung, spleen, and a pool of tracheobronchial, mesenteric and inguinal lymph nodes.

### Bacteriology

Tissue samples from animals with CNS signs and gross lesions in various tissues were submitted for bacteriological analysis to rule out or confirm bacterial infections as appropriate.

### Histopathology

Brain, spinal cord, tonsil, thymus, lung, heart, spleen, lymph nodes, spleen, liver, kidney and intestinal organs samples of the study animals were collected in 10% formalin and submitted to ISU-VDL for microscopic evaluation. Microscopic lesions will demonstrate pathogenicity of the isolates evaluated.

### Results

### Clinical Observations

Animals were observed daily for the duration of the study and monitored for the development of clinical signs that included neurological signs, mortality, respiratory disease, and diarrhea and measurement of rectal temperature as indicated in study design section.

### Rectal Temperatures

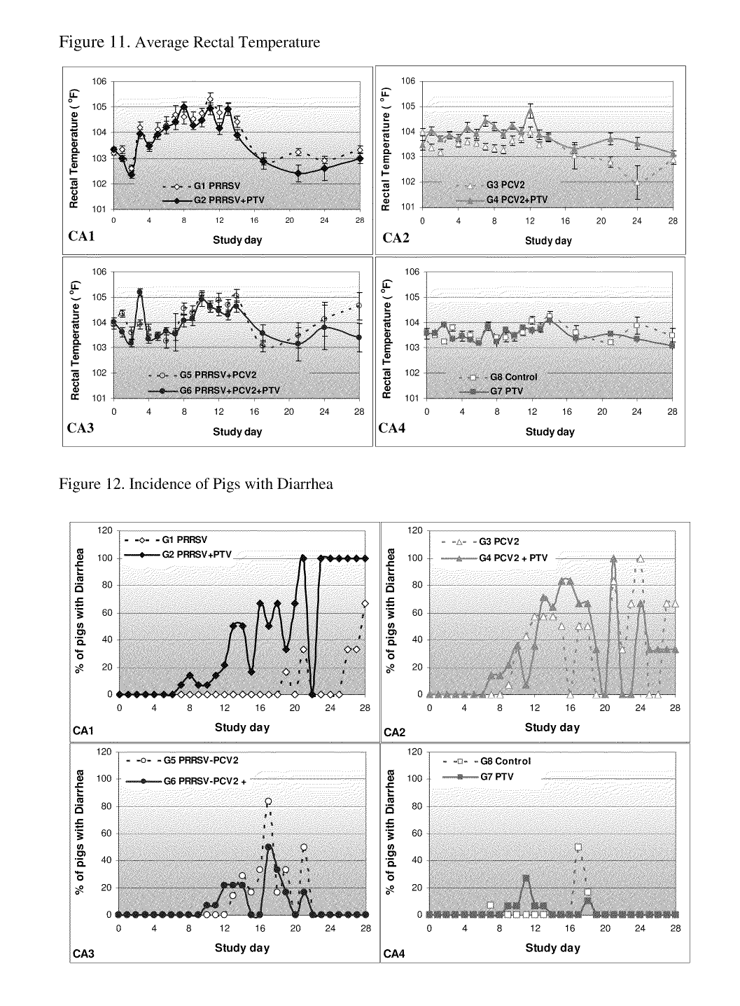

The mean rectal temperatures measured in Fahrenheit (° F.) for all groups 1 to 8 are shown in FIG. 11. The average temperature expressed in degree Fahrenheit (° F.) for each group was calculated at the indicated study days. The error bars represent the standard error of the means. The label in the lower left corner indicates the room where the two groups were located. Solid lines indicate the group in each room directly inoculated with PTV.

The results show that pigs exposed to PRRSV (groups 1, 2, 5 & 6 in rooms CA1 and CA3) tended to have higher rectal temperature (≧104.5° F.) than pigs not exposed to PRRSV. The increase in rectal temperature was more consistently detected between study days 6 to 14. At least 50% of PRRSV-infected pigs had temperatures >104.5° F. by day 7 post-infection and by day 14 90-100% had developed high temperatures. In contrast the pigs infected or exposed to PTV alone (groups 7 and 8) had temperatures within the normal range during the study. The pigs infected with PCV2 and one week later with PTV (group 4) tended to have a slight increase in temperature relative to groups 3, 7 and 8 but lower levels than the PRRSV-infected (groups 1, 2, 5 and 6) pigs. It was not possible to clearly confirm the effect of PTV on the rectal temperature in the PRRSV and/or PCV2-infected pigs seen in the previous study due to the cross-contamination reported above.

### Neurological Signs

Neurological signs characterized by mild depression were mostly observed in PRRSV-infected pigs that were intravenously exposed with PTV one week later (group 2) with 50% affected at some time during the study: One pig developed severe signs by study day 4 (unable to stand and shaking and turned into mild depression) and recovered by day 12 and the other six pigs developed depression 3 to 10 days post-PTV exposure with two of these pigs recovering next day of the onset whereas in the other pigs the signs persisted for two to 4 days. Mild neurological signs were observed in only one pig in groups 1 (at day 12), group 3 (at days 24-26), group 5 (at days 19 and 20), and group 7 (at days 11-14), or two pigs in group 6 (one at days 13 and 14 just prior to euthanasia at the other at day 19 the day before its death). None of the pigs in groups 4 and 8 developed apparent neurological signs.

### Diarrhea

FIG. 12 shows the incidence of pigs affected per group. The proportion of pigs per group with diarrhea at each study day is indicated in percentage based on the total of number of pigs alive at each time point. For study days 1-14 n=14 except for group 7 n=15. For study days 15-21 n=6 except for group 7 n=10. For study days 22-28 n=3 except for G7 n=5. Each graph represents the data of the two groups allocated in the same room. Solid lines indicate the group in each room directly inoculated with PTV.

In this study, diarrhea was a predominant clinical observation, especially in groups 2, 3, 4, 5, and 6. Severe diarrhea that was intermittent or persisted throughout the study in most of the affected pigs was observed in groups 2, 3 and 4 with largest number of pigs affected at study days 13 and 14 whereas in groups 5 and 6, fewer pigs had a delayed onset of severe diarrhea. The data show that pigs intravenously inoculated with PTV tended to develop diarrhea early after infection, whereas the pigs in the same room that became sentinels had a delayed onset. Prior exposure to PRRSV and PCV2 appeared to increase the incidence of the affected pigs. Interestingly, the effect of single infection with PRRSV or PCV2 prior to PTV exposure on the incidence, severity and duration of diarrhea was relative higher than that induced by dual PRRSV+PCV2 infection. However the effect of PCV2 infection was more severe than that of PRRSV in the sentinel pigs.

### Respiratory Signs

Respiratory signs were observed only transiently in a relatively few pigs per group and they were mainly characterized by mild cough and/or rapid respiration. The proportion of pigs per group with clinical respiratory signs at each study day is indicated in percentage based on the total of number of pigs alive at each time point. For study days 1-14 n=14 except for group 7 n=15. For study days 15-21 n=6 except for group 7 n=10. For study days 22-28 n=3 except for G7 n=5. Each graph represents the data of the two groups allocated in the same room. Solid lines indicate the group in each room directly inoculated with PTV. Only one pig in group 2 had severe respiratory signs at study days 11, 13 and 14.

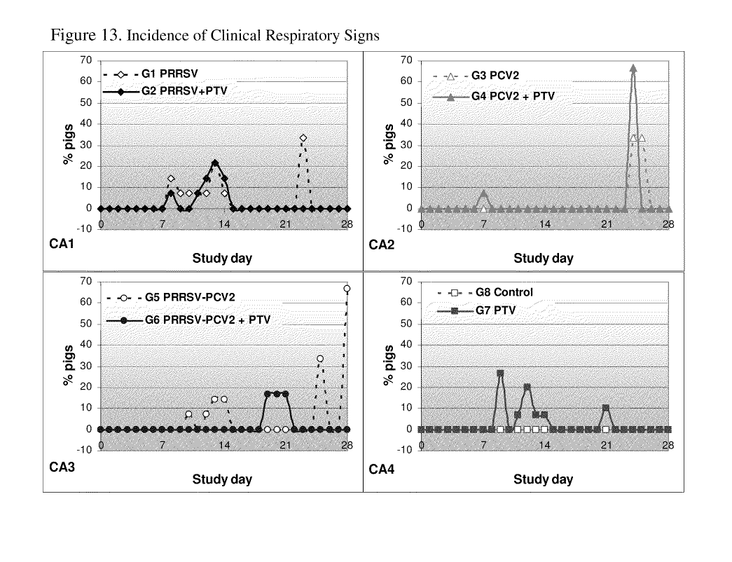

The incidence of the respiratory signs over time during the duration of the study is illustrated in FIG. 13. The majority of the affected pigs in groups 1, 2 and 7 developed clinical respiratory signs between study days 7-14. However, some of the remaining pigs in groups 1, 3-7 developed transient respiratory abnormalities at later time points.

Contrary to what was expected, the highest incidence of respiratory signs were observed in the pigs only intravenously exposed to PTV (group 7) with approximately 53% of pigs affected at least one day during the study whereas only 33% of the PRRSV-infected pigs showed respiratory signs and even a lower percentage of affected pigs was observed in the dual PRRSV+PCV2 infected pigs.

### Mortality

Three pigs died during the study. Two of the pigs were in group 5 and one pig in group 6. One of the pigs from group 5 died at study day 7 while being examined for rectal temperature, did not have apparent clinical signs but the rectal temperature recorded for this pig at day 7 was only 94.1° F. and upon necropsy lung lesions consistent with PRRSV infection and abdominal fluid were observed. The other pig in group 5 died at study day 25 with no apparent clinical signs the day prior to its dead but had episodes of diarrhea between study day 12 and 18 with three of them rated as severe and necropsy examination of this pig revealed severe lung lesions comprising 100% of the organ. The pig in group 6 died at study day 20. This pig in contrast, was showing respiratory signs, had severe diarrhea and was unable to stand and walk the day prior to be found dead. Necropsy examination showed severe lung lesions and gastric ulcer and was found with microscopic mild CNS and moderate lung lesions.

### Necropsy Findings

Necropsy was done on any pig that died outside of the scheduled times. All other necropsy examinations were performed following euthanasia as scheduled on eight pigs from each group (except group 7) on study day 14, three pigs on study days 21, and 28. For group 7 five pigs were euthanized at each schedule day. The most consistent finding was interstitial pneumonia in the PRRSV-exposed pigs and the gross lung lesions as noted below. However in a variable percentage of pigs per group, microscopic lesions in other organs were observed at all the three necropsy times.

### Gross Lung Lesions

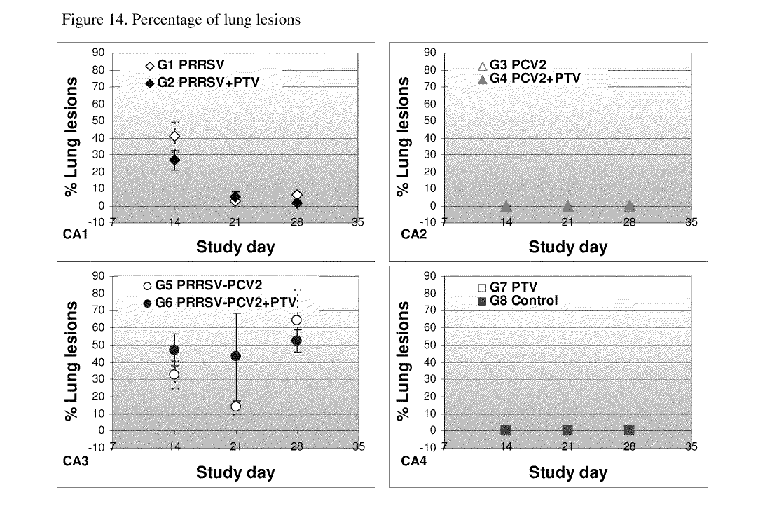

Lung lesions were observed in all PRRSV-exposed pigs (groups 1, 2, 5 and 6) at day 14 with variable levels of lung involvement. FIG. 14 shows the average % of lung lesions per group at each euthanasia day. The average of the lung lesions per group at the indicated necropsy times are shown in percentage based on relative area of lung affected per pig. Error bars represent the standard error of the mean. Each graph represents the data of the two groups allocated in the same room. Filled symbols indicate the group in each room intravenously inoculated with PTV.

In this study co-infection with PCV2 or exposure to PTV did not seem to significantly increase the level of gross lung lesions in the PRRSV-infected pigs at day 14. However, PCV2 co-infection tended to prolong the severity of gross lung lesions since the dually infected pigs (groups 5 and 6) euthanized at later days had higher percentage of lung lesions compared to the pigs in the absence of PCV2 (groups 1 and 2).

The observation that two of the three pigs in group 6 (intravenously exposed to PTV) had more extensive lung lesions that the three pigs in group 5 (sentinel pigs) euthanized at day 21 was interesting and consistent with previous findings suggesting that PTV may influence the severity of the disease, which may be related to PTV dose and/or time of exposure.

At day 28 all the PRRSV-PCV2-infected pigs had ≧39% lung involvement. In contrast, the pigs that were only infected with PRRSV and then exposed directly or indirectly with PTV (groups 1 and 2) had no or minimal (<10%) lung lesions at day 21 and 28. None of the PCV2 exposed pigs in groups 3 and 4 nor the control pigs in group 8 had lesions at the three necropsy dates whereas in group 7 negligible (≦1%) lung lesions were observed in one pig at study day 14 and in 2 pigs at study day 21.

### Microscopic Lesions

A summary of the number of pigs with microscopic lesions for each group is indicated in Table 12.

TABLE 12
Distribution of animals with microscopic lesions

| | | | Spinal | | | | | | Lymphoid |
|---|---|---|---|---|---|---|---|---|---|
| | n | Brain | cord | Lung | Heart | liver | kidney | Spleen | depletion |
| Group - Day 14 | | | | | | | | | |
| G1 PRRSV | 8 | 3 | 1 | 3 | 3 | 4 | 2 | 3 | 3 |
| G2 PRRSV + PTV | 8 | 2 | 0 | 2 | 2 | 2 | 2 | 1 | 2 |
| G3 PCV2 | 8 | 2 | 0 | 3 | 2 | 2 | 1 | 1 | 2 |
| G4 PCV2 + PTV | 8 | 1 | 0 | 3 | 1 | 3 | 1 | 2 | 2 |
| G5 PRRSV-PCV2 | 8 | 2 | 0 | 7 | 1 | 2 | 0 | 3 | 4 |
| G6 PRRSV-PCV2 + | 8 | 3 | 0 | 6 | 3 | 1 | 0 | 2 | 4 |
| PTV | | | | | | | | | |
| G7 PTV | 5 | 0 | 0 | 1 | 3 | 2 | 2 | 0 | 0 |
| G8 Control | 8 | 2 | 0 | 2 | 3 | 2 | 3 | 1 | 1 |
| Group - Day 21 | | | | | | | | | |
| G1 PRRSV | 3 | 0 | 0 | 3 | 0 | 0 | 0 | 0 | 1 |
| G2 PRRSV + PTV | 3 | 2 | 1 | 2 | 0 | 2 | 0 | 1 | 1 |
| G3 PCV2 | 3 | 1 | 0 | 0 | 1 | 1 | 1 | 0 | 0 |
| G4 PCV2 + PTV | 3 | 0 | 0 | 1 | 0 | 1 | 0 | 1 | 1 |
| G5 PRRSV-PCV2 | 3 | 0 | 0 | 0 | 0 | 0 | 1 | 0 | 0 |
| G6 PRRSV-PCV2 + | 3 | 1 | 0 | 2 | 0 | 3 | 0 | 0 | 1 |
| PTV | | | | | | | | | |
| G7 PTV | 5 | 2 | 0 | 2 | 1 | 1 | 0 | 0 | 1 |
| G8 Control | 3 | 0 | 0 | 0 | 0 | 0 | 0 | 0 | 0 |
| Group - Day 28 | | | | | | | | | |
| G1 PRRSV | 3 | 1 | 1 | 2 | 2 | 2 | 0 | 1 | 1 |
| G2 PRRSV + PTV | 3 | 2 | 1 | 1 | 2 | 2 | 0 | 0 | 2 |
| G3 PCV2 | 3 | 2 | 1 | 1 | 2 | 2 | 3 | 0 | 1 |
| G4 PCV2 + PTV | 3 | 1 | 0 | 2 | 0 | 2 | 2 | 0 | 1 |
| G5 PRRSV-PCV2 | 3 | 1 | 0 | 2 | 1 | 2 | 1 | 0 | 1 |
| G6 PRRSV-PCV2 + | 5 | 1 | 0 | 1 | 2 | 1 | 1 | 0 | 0 |
| PTV | | | | | | | | | |
| G7 PTV | 3 | 2 | 0 | 4 | 2 | 3 | 2 | 2 | 2 |
| G8 Control | 0 | 0 | 0 | 0 | 0 | 0 | 0 | 0 | 0 |

### CNS Lesions

Microscopic lesions in the brain and spinal cord in most of the affected pigs were characterized by mild multifocal nonsuppurative perivascular infiltrates. Three pigs one in each of groups 1, 2 and 3 had moderate brain lesions, and one in group 4 had meningitis. The highest incidence of CNS microscopic lesions was observed in groups 2, 3, and 6, and the lowest in groups 4 and 8. When comparing the clinical CNS scores with the results of the microscopic analysis it was found that out of 12 pigs that had clinical CNS signs, only four (three in group 2 and one in group 6) also had microscopic CNS lesions. All other 8 pigs with clinical CNS signs did not have CNS microscopic lesions. Conversely, out of 31 pigs that had microscopic lesions in the CNS only the four 4 pigs indicated above had CNS signs prior to and or at the necropsy day whereas all other 27 pigs with microscopic CNS lesions did not show obvious CNS clinical sings during the observation period.

### Lung Lesions

The microscopic lesions detected in the majority of the lung samples consisted of mild to moderate diffuse (in few cases mild to severe patchy) non-suppurative interstitial pneumonia. One pig in group 2 had mild interlobular edema and fibrosis. The pig that died at day 7 in group 5 had congestion and hyperemia in pulmonary vessels, atelectasis and interstitial pneumonia. Table 13 shows the percentage of pigs with microscopic lung lesions as compared to the percentage of pigs with gross lung lesions and respiratory clinical signs.

TABLE 13
Percentage of pigs per group affected with lung lesions or clinical signs

| | | % of pigs affected per group | | |
|---|---|---|---|---|
| | Number of | Clinical | Gross | |
| | pigs per | Respiratory | Lung | Microscopic |
| Group | group | Signs | Lesions | Lung Lesions |
| G1 PRRSV | 14 | 35.7 | 85.7 | 71.4 |
| G2 PRRSV + PTV | 14 | 35.7 | 92.9 | 35.7 |
| G3 PCV2 | 14 | 7.1 | 0.0 | 28.6 |
| G4 PCV2 + PTV | 14 | 21.4 | 7.1 | 42.9 |
| G5 PRRSV + PCV2 | 15 | 40.0 | 100.0 | 60.0 |
| G6 PRRSV + | 14 | 14.3 | 100.0 | 64.3 |
| PCV2 + PTV | | | | |
| G7 PTV | 15 | 53.3 | 20.0 | 46.7 |
| G8 Control | 14 | 0.0 | 0.0 | 14.3 |

Bold numbers indicate groups with >50% of pigs affected.

Similar to what was observed for the CNS parameter, there was not complete correlation of the clinical respiratory signs with either gross or microscopic lung lesions. Only 10 out of the pigs that had clinical respiratory signs also had detectable gross and microscopic lesions whereas of the 58 pigs with gross lung lesions approximately 53% of those pigs also had microscopic lesions but only 33% of them showed clinical signs. The discrepancy between macroscopic and microscopic lesions may have been the result of improper selection of tissue sections for histopathology whereas those related to the clinical signs may be due to differential onset for the parameters analyzed.

### Lymphoid Tissues

The most consistent finding was mild lymphoid depletion in various lymphoid tissues in the number of pigs indicated in Table 13.

### Heart

Heart lesions were mostly characterized by mild multifocal nonsuppurative perivascular infiltrates in all the pigs indicated in Table 13 except in two pigs that had moderate multifocal nonsuppurative mycocarditis, one pig in group 1 and one pig in group 6 both at day 14.

### Liver

Mild, multifocal, periportal, mixed hepatic inflammation was the predominant feature in the affected pigs except for two pigs that had moderate multifocal, periportal inflammation, one in group 1 and one in group 4.

### Kidney

The pigs affected as indicated in Table 13 had multifocal nonsuppurative interstitial nephritis.

### Average Daily Weight Gain

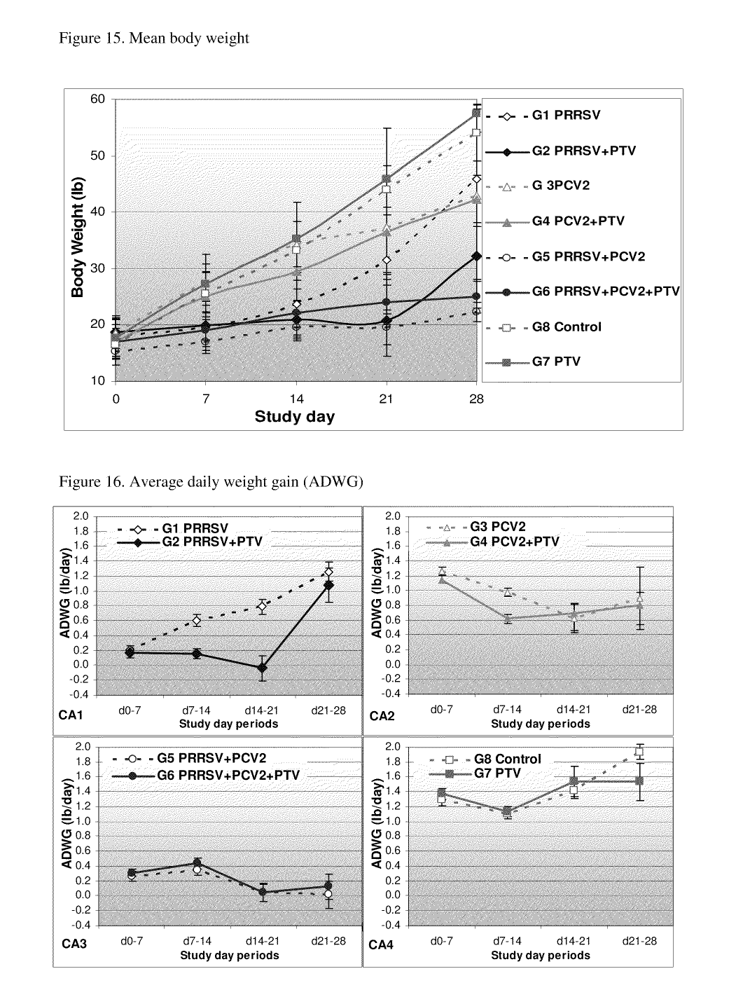

All pigs were weighed on study day 0 and day 7 and at the scheduled euthanasia day to determine the average daily weight gain (ADWG) for each animal. The ADWG during each week period was calculated by subtracting the weight on the earliest study day of each week period from the weight at the last day of each week period or necropsy day if different and dividing by the number of days between the measurement points. FIG. 15 shows the average body weight per group and FIG. 16 the ADWG for the indicated time periods. Data represent the average weight per group at the indicated study days and error bars indicate the standard deviation. Solid lines indicate the group in each room intravenously inoculated with PTV.

The results in FIG. 10 show the effect of the different treatments on body weight over time. Whereas the pigs that were only directly or indirectly exposed to PTV (groups 7 and 8, respectively) had an steady increase in body weight during the duration of the study, the pigs in all other groups had in general lower body weight. The pigs co-infected with PRRSV and PCV2 at day 0 had the worst performance, followed by the PRRSV-infected pigs that were intravenously exposed to PTV. The effect on weight gain was best assessed by determining the average daily weight gain for each pig.

The ADWG was calculated for each pig during the indicated weekly study periods. The data shown is the mean of the ADWG for each group and errors bars are the standard error of the means. For study week period day 0 to day 7 n=14 for groups 1, 2, 3, 4, 6 and 8 and for groups 5 and 7 n=15. For study week period day 7 to day 14 n=14 for groups 1, 2, 3, 4, 5, 6 and 8 and for group 7 n=15. For study week period day 14 to day 21 n=6 for groups 1, 2, 3, 4, 5, 6 and 8 and for group 7 n=10. For study week period day 21 to day 28 n=3 for groups 1, 2, 3, 4, 5, 6 and 8 and for group 7 n=5. Each graph represents the data of the two groups allocated in the same room. Solid lines indicate the group in each room intravenously inoculated with PTV.

The data clearly show that PRRSV infection had the most detrimental effect on the ADWG during the first week following infection Animals that were only infected with PRRSV tended to slowly recover over time but they did not reach the levels of gain seen in pigs of groups 7 and 8. Co-infection with PCV2 maintained and prolonged the detrimental effect induced by PRRSV on the ADWG with these animals having a minimal weight gain and even losing weight during the last two weeks of the study. The effect of PTV on the ADWG was best appreciated during the first two weeks following PTV intravenous exposure (study days 7 to 14 and days 14 to 21) in the PRRSV infected pigs. Whereas the PRRSV only infected group tended to increase the rate of weight gain, the PRRSV-infected pigs that were intravenously exposed one week later with PTV overall had minimal weight gain with some pigs losing weight during these two week periods, and although the animals appear to recover and gain weight during the last week of the study they did not reach the levels seen in the control pigs (Group 8). The effect of PTV on ADWG was also observed in the PCV2-infected pigs during the first week following PTV exposure. PTV by itself did not seem to have a detrimental effect when compared to the control pigs. The rate of weight gain observed in the control pigs of this study was equivalent to that observed in the control pigs in the study described in Examples 1 with conventional pigs.

### Virus Isolation

Recovery of viruses from the infected pigs was confirmed based on CPE and staining with virus-specific antibodies for PRRSV in AK-MA104 cells, and for PTV in PKWRL cells. Isolation of PCV2 was based on specific staining in VIDO-R1 cells. Table 14 summarizes the results of virus isolation for tissue samples. The results of the pigs that died earlier were added to the results obtained in the subsequent scheduled necropsy date.

TABLE 14
Summary of virus isolation from tissue samples

| | Virus isolated | | | | | | | | |
|---|---|---|---|---|---|---|---|---|---|
| | PTV | | | PRRSV | | | PCV2 | | |
| | Necropsy day | | | | | | | | |
| | 14 | 21 | 28 | 14 | 21 | 28 | 14 | 21 | 28 |
| G1 PRRSV | 3 | 1 | 3 | 4 | 0 | 0 | 0 | 0 | 0 |
| G2 PRRSV + PTV | 5 | 3 | 0 | 1 | 1 | 0 | 0 | 0 | 0 |
| G3 PCV2 | 8 | 3 | **0 | 0 | 0 | 0 | 7 | 2 | 0 |
| G4 PCV2 + PTV | 8 | 2 | 1 | 0 | 0 | 0 | 8 | 3 | 3 |
| G5 PRRSV-PCV2 | 3 | 2 | 1 | 3 | 2 | 3 | 8 | 3 | 3 |
| G6 PRRSV-PCV2 + | 4 | 1 | 2 | 1 | 3 | 3 | 8 | 3 | 3 |
| G7 PTV* | 4 | 1 | 0 | 0 | 0 | 0 | 0 | 0 | 0 |
| G8 Control | 2 | 2 | 0 | 0 | 0 | 0 | 0 | 0 | 0 |
| n* (pigs per group) | 8 | 3 | 3 | 8 | 3 | 3 | 8 | 3 | 3 |

PRRSV and PTV isolation based on CPE and IFA. PCV2 isolation based on IFA.
Group (G1 to G8) treatment designation based on experimental infection as indicated in the experimental design. Pig in groups 2, 4, 6 and 7 were intravenously exposed to PTV.
Numbers represent number of positive pigs at the indicated necropsy day.
*n = 5 for group 7 at all three necropsy days.
**One pig in G3 was positive for PTV in the day 14 serum sample but negative in the tissue samples at day 28.

### PTV Isolation

The data in Table 15 shows that a variable proportion of pigs from all groups were positive for PTV isolation. These results indicate that the separation between treatment groups allocated in the same room as described in the study design was not sufficient to prevent cross-contamination and therefore compromising the outcome of the study. Based on these findings, no clear distinction can be made between the two groups allocated in the same room other than the groups indicated with PTV in the subsequent results correspond to groups that were intravenously inoculated with PTV. It may be speculated that the pigs that became sentinels may have been exposed to lower levels of PTV within a week after the IV-inoculated pigs. Nevertheless, it is interesting to note that PCV2 and/or PRRSV infection prior to PTV exposure tended to increase the PTV dissemination among and within each pig because a larger number of pigs were PTV positive in these groups than in pigs only exposed to PTV. PCV2 by itself had an apparently greater effect on the PTV infection: a larger number of pigs in the two groups infected with PCV2 (groups 2 and 4) were positive for PTV isolation from various tissues and a higher PTV recovery from the CNS was also observed in pigs of groups 3 and 4 compared to the results of PTV isolation from the other six groups (Table 12).

TABLE 15
PTV isolation from the CNS tissues

| Virus isolated | PTV in CNS | | |
|---|---|---|---|
| Necropsy day | 14 | 21 | 28 |
| G1 PRRSV | 3 | 1 | 2 |
| G2 PRRSV + PTV | 4 | 1 | 0 |
| G3 PCV2 | 8 | 2 | 0 |
| G4 PCV2 + PTV | 5 | 2 | 1 |
| G5 PRRSV-PCV2 | 3 | 0 | 0 |
| G6 PRRSV-PCV2 + PTV | 1 | 0 | 2 |
| G7 PTV* | 4 | 0 | 0 |
| G8 Control | 1 | 0 | 0 |
| Number of pigs per group (n*) | 8 | 3 | 3 |

Numbers indicate the number of pigs that were positive for PTV isolation from the brain in the corresponding treatment groups as described in experimental design.
*n = 5 for group 7 at all three necropsy days.

### PRRSV Isolation

Viremia was confirmed by virus isolation in all PRRSV-infected pigs in groups 1, 2, 5 and 6 at 7 days post-inoculation. PRRSV was also isolated from serum at day 14 from all except one of the PRRSV-inoculated pigs. PRRSV was also isolated from the lung in the number of pigs indicated in Table 3 and few of the pigs were also positive in the lymph node, tonsil or spleen. The serum and tissue samples from pigs in all other groups were negative for PRRSV isolation. In the group that was co-inoculated with PCV2 and exposed to PTV more pigs were PRRSV positive in the lung by virus isolation at later days post-exposure compared to the pigs that were only inoculated with PRRSV at day 0.

### PCV2 Isolation

PCV2 was only isolated from pigs that were inoculated with PCV2. The virus was recovered from various tissues including tonsil, lymph nodes, and spleen and less consistently from the brain. Intravenous PTV exposure appear to increase the detectable levels of PCV2 as basically all pigs were positive for PCV2 at all three necropsy dates whereas the sentinel pigs that were most likely exposed to PTV by oronasal route appeared to clear the PCV2 earlier after inoculation.

### PRRSV and PCV2 Viremia by Real-Time PCR Analysis

The PRRSV and PCV2 viremia levels were determined by Real-Time PCR and the results shown in FIG. 21. The levels of PRRSV (A) and PCV2 (B) were determined in the serum samples for all pigs in the study at the indicated study days. Data for the groups that were found positive for the corresponding viral RNA are shown. Data is expressed as the mean copy number of the indicated virus calculated based on a standard curve included in the assay. Error bars are the standard error of the means for each group. Solid lines indicate the groups intravenously exposed to PTV.

The results show that PRRSV viremia reached high levels as early as 3 days post-infection and peaked around 7 days post-infection. PRRSV RNA levels tended to decrease over time. However, PCV2 co-infection tended to prolong the high PRRSV viremic levels in the infected pigs. Similarly, PCV2 viremic levels were enhanced in the animals that were co-infected with PRRSV (groups 5 and 6). Interestingly, higher PCV2 levels were also observed in animals of group 4 compared to those in group 3, suggesting that PTV may also have some modulatory effect on PCV2 viremia that may depend on PTV levels and/or time of PTV infection with respect to PCV2 exposure.

The Real-Time PCR analyses confirmed the virus isolation results in that only the animals that were experimentally inoculated with PRRSV were positive for PRRSV RNA and only the pigs that were experimentally inoculated with PCV2 were positive for PCV2 DNA. All other pigs were negative for PRRSV or PCV2 nucleic acids in the serum samples tested.

### Serology

### Pre-Screening for PRRSV, PCV2 and PTV Antibodies

Serum samples collected at birth were analyzed for antibodies to PRRSV, PCV2 and PTV. Results of the analyses show that all pigs were negative for antibodies to the indicated viruses and therefore suitable for the study.

### PTV Serology

The serological response to PTV was analyzed by virus neutralization antibody (VNA) test in all pigs of the study and the results are shown in FIG. 22. Anti-PTV antibodies were measured by virus neutralization assay (VNA). Titers are expressed as the reciprocal of the last serum dilution inhibiting PTV-induced CPE. Data represent the mean antibody titers for each group. Each graph represents the data of the two groups allocated in the same room.

The serological analysis for PTV confirmed the virus isolation results in that the groups not directly inoculated with PTV were indirectly exposed to PTV from the shedding of the intravenously PTV-exposed pigs located in the same room. PTV exposure levels in the sentinel pigs were sufficient to induce antibody responses. The data shown for pigs in room CA4 clearly demonstrate that PTV exposure induced a rapid and high level antibody response in the absence of other virus infections. All the PTV-infected pigs in group 7 had relatively high anti-PTV antibody VNA titers one week after intravenous exposure. The antibody titers peaked by day 21 (14 days post-PTV exposure) and maintained the high levels to the end of the study, except for one of the five pigs which antibody titers slightly decreased by study day 28. Analysis of the anti-PTV antibody response in the sentinel pigs (group 8) exposed to PTV most likely by the oronasal route showed a similar kinetics but with an apparent onset delay of one week relative to group 7.

The antibody response to PTV was inhibited by other viral infections with dual PRRSV+PCV2 co-infection having the greatest inhibitory effect. However, PRRSV by itself had an inhibitory effect by delaying the antibody response to PTV. PCV2 pre-exposure also affected the serologic response to PTV at much lesser extend than PRRSV or the dual PRRSV+PCV2 infections. The serological analysis also showed low levels of neutralizing antibodies in a small proportion of pigs at study day 7 and even in a fewer number with lower titers at study day 0 in samples collected prior to the experimental PTV exposure, suggesting some potential exposure to low levels of environmental PTV.

### PRRSV Serology

FIG. 23 shows the serological response to PRRSV. Data is expressed as the mean of S/N values for each group. Solid lines indicate the animals that were directly inoculated with PTV by intravenous route. The kinetics of the anti-PRRSV antibody response was equivalent in all four PRRSV-infected groups until day 17. PCV2 and PTV exposure tended to slightly decrease the anti-PRRSV antibody levels at later time points. Seroconversion to PRRSV was demonstrated in all pigs that were infected with PRRSV in groups 1, 2, 5 and 6. No other pig developed antibodies to PRRSV during the duration of the study.

### PCV2 Serology

Antibody responses to PCV2 are illustrated in FIG. 24. PCV2 antibodies were determined by indirect fluorescence antibody assay (IFA). Titers were expressed as the reciprocal of the last dilution with specific fluorescence detected. Results for the groups infected with PCV2 are shown. Solid lines indicate the animals that were directly inoculated with PTV by intravenous route.

Antibody responses to PCV2 in the PCV2-infected pigs developed relatively slower when compared to the antibody responses induced by PRRSV or PTV. All other pigs, except for one pig in group 7, had undetectable levels of anti-PCV2 antibodies. The result for the pig in group 7 was completely unexpected since this group was not exposed to PCV2, none of the other pigs in this group had any anti-PCV2 antibodies and all pigs in group 7 were negative for virus isolation and did not have detectable levels of PCV2 DNA in the serum as determined by real-time PCR.

Nevertheless, the serological analysis showed that pigs co-infected with PRRSV had lower anti-PCV2 antibody titers as detected by IFA. It was also interesting to note that pigs directly inoculated with PTV had a lower serological response to PCV2 than pigs indirectly exposed to PTV.

### Conclusions

The major goals of the study were to confirm the relevance of PTV in the respiratory disease induced by PRRSV or PRRSV-PCV2 infections and determine whether in the absence of maternal immunity PTV by itself or in combination with PRRSV and/or PCV2 pre-exposure induced CNS clinical signs. Due to the unexpected cross-contamination that occurred as demonstrated by virus isolation results and serological analysis to PTV, it was not possible to conclusively determine to what extent PTV influences the severity of the respiratory disease induced by PRRSV or PRRSV-PCV2 infections. Similarly, the role of PTV, PRRSV and PCV2 in the CNS clinical signs could not be assessed with confidence. Nevertheless, the results of this study provided important insights in the pathogenesis of PTV, PRRSV and PCV2 in a disease complex as discussed below.

This study further demonstrated that PTV is a highly infectious virus that disseminates very quickly in susceptible pigs. This was evidenced by a high rate of recovery of PTV by virus isolation from several tissues. In this study it was clear that CDCD pigs were highly susceptible to PTV infection with 80% of the pigs positive for PTV isolation from various organs including the brain and spinal cord one week after direct PTV exposure with no other viral infection, whereas in the previous study with conventional pigs that had some level of maternal immunity, PTV could only be recovered from the tonsil in 18% of the pigs exposed only to PTV. This study also shows that in the absence of other infections, PTV appears to be cleared relatively quickly with the exception of one pig that was PTV positive in the lung 14 days post-infection, PTV could not be isolated in pigs euthanized 14 or 21 days post-PTV exposure. This finding is consistent with Example 2, in which the PTV-infected CDCD pigs were negative for PTV isolation at 21 days post-infection. It was also evident that in contrast to PRRSV, blood was less sensitive for PTV detection by virus isolation probably due to the rapid development of high levels of anti-PTV neutralizing antibodies in the infected pigs. It is also important to note that despite the high level of neutralizing antibodies detected at seven days post-PTV intravenous exposure, the virus was still present in multiple tissues at levels that could be detected by virus isolation in a high proportion of infected pigs. These are relevant findings to consider in the design of future studies to further address the pathogenicity of PTV.

Similar to the previous study with few CDCD pigs (Example 2), this study also showed that in the absence of other infections, the CDCD pigs developed a relatively rapid antibody response with high levels of neutralizing antibodies as early as 7 days after intravenously PTV exposure. In contrast, prior exposure to PRRSV and/or PCV2 negatively affected the serological response to PTV. However, the negative effect on the anti-PTV antibody response did not seem to correlate with the level of systemic PTV dissemination. Whereas the dual infection with PRRSV and PCV2 had the greatest effect on the anti-PTV antibody response, and the inhibitory effect on the anti-PTV antibody response induced by single infection with PRRSV was greater than that induced by PCV2 single infection, PCV2 by itself appeared to influence to a greater extend the dissemination of PTV in the infected pigs. This was evidenced by the larger number of pigs positive for PTV isolation from multiple tissues in the PCV2 infected pigs whether directly or indirectly exposed to PTV. PCV2 may also influence the PTV viremia because the only pigs that were positive for PTV in the serum were pigs in group 3 that were exposed to PCV2 and one week later intravenously exposed to PTV. These results point out PCV2 as an important factor in the dissemination of PTV and suggest that an immunological function affected by PCV2 infection may be critical for early PTV clearance in swine.

The lack of maternal immunity combined with the intravenous exposure to a high dose of PTV may explain the extent of PTV isolation from multiple tissues in four out of five pigs early after single PTV infection and the rapid and high neutralizing antibody responses induced in these pigs may explain why PTV levels were dramatically reduced to undetectable levels at later times points. Despite the systemic dissemination of PTV in the susceptible pigs, the normal immune response appear to be sufficient to clear or reduce the PTV infection to levels that prevent or reduce to a great extent the pathology of the PTV strain used in this study, given that these animals gained a normal body weight, did not develop significant clinical neurological signs and only very few pigs develop mild diarrhea when compared to other treatments groups. The PTV used in the study, however, appeared to induce a mild and transient respiratory distress in the conditions evaluated in group 7. The transmission of PTV from the single PTV-infect pigs to the control pigs was sufficient to infect pigs to levels that were detected by virus isolation in approximately 28% of the pigs and to induce a vigorous and rapid antibody response in all the pigs but not enough to have a significant clinical effect.

Although the role of PTV in the respiratory disease induced by PRRSV and/or PCV2 could not be clearly demonstrated based on respiratory scores, overall lung lesions or increase in body temperature, PTV did increase the severity of gross lung lesions in the dually PRRSV/PCV2 infected pigs, effect observed at two weeks post-PTV intravenous exposure. In addition, it was also apparent that PTV tended to have a negative effect in the infected pigs previously exposed with PRRSV and/or PCV2 by prolonging the PRRSV and PCV2 viremia as detected by Real-time PCR and delaying and decreasing the level of anti-PCV2 antibody response. More evident was the effect of PTV infection on the reduction in average daily weight gain induced by PRRSV and/or PCV2, which was clearly seen during the first two weeks following intravenously PTV exposure. This is an important economical parameter to be considered in further evaluations.

The second goal was to determine whether in the absence of maternal anti-PTV antibodies, PTV infection may induce CNS disease by itself or as a consequence of PRRSV and/or PCV2 pre-exposure. Similar to the previous study, only mild neurological signs characterized by depression were observed in most of the pigs affected and only two pigs developed severe CNS signs. The higher incidence of CNS signs and microscopic lesions in the CNS tissues observed in group 2 suggests that PRRSV may be a predisposing or co-factor for PTV-induced CNS disease. Further analysis with proper controls will provide more conclusive evidence of the role of PRRSV in the CNS disease induced by PTV. However, based on the severity of neurological signs observed in the original pigs from which the PTV, PRRSV and PCV2 viruses were isolated and the mild neurological manifestations observed in the present study and despite the development of microscopic CNS lesions and/or PTV in the CNS tissues, it can be hypothesized that other unknown factors may be required for the viruses evaluated to induce severe CNS disease.

### Example 4

### Objective

(1) To evaluate a mixed challenge model of PCV2, PRRSV and PTV in CDCD pigs vaccinated with PTV, PCV2 or PRRSV.

### Materials and Methods

### Experimental Design

For the experiment, 84 CDCD pigs were randomly allocated into 9 treatment groups as indicated in Table 16. Treatment groups were either vaccinated or non-vaccinated and either challenged singularly or in combinations with PRRSV, PCV2 and PTV (groups 1-8) or left as negative controls (group 9). Table 17 provides the description of challenge materials and vaccines used in the study. All animals were observed daily for abnormal clinical signs; rectal temperatures were taken daily. All animals were weighed and bled on study day 0, 28, 35 and 42. The study was terminated at day 42.

TABLE 16
Study Design

| | | | | | | | | | PTV | | |
|---|---|---|---|---|---|---|---|---|---|---|---|
| | | | | | | | | | Challenge | | |
| | | | | | PRRSV | | | | 6.5log | | |
| | | | Vaccination | | 4.5 log | | PCV2 | | TCID50/mL | | |
| | | | IM, 1 mL | | TCID50/ml | | Challenge | | D35 | | |
| | | | D0 | D14 | Challenge D28 | | | Dose | | Dose | |
| Gp | n | rm | Rt side | Left side | Route | Dose Size | Route | Size | Route | Size | Necropsy |
| 1 | 10 | 1 | — | — | IN | 2 mL | — | — | — | — | D42 |
| 2 | 10 | 2 | — | — | IN | 2 mL | | | IV | 2 mL | D42 |
| 3 | 10 | 2 | kPTV | kPTV | IN | 2 mL | | | IV | 2 mL | D42 |
| 4 | 10 | 4 | — | — | IN | 2 mL | IN | 2 mL | — | — | D42 |
| 5 | 10 | 3 | — | — | IM | 2 mL | IN | 2 mL | IV | 2 mL | D42 |
| 6 | 10 | 3 | Circo | — | IM | 2 mL | IN | 2 mL | IV | 2 mL | D42 |
| 7 | 10 | 3 | kPTV | kPTV | IM | 2 mL | IN | 2 mL | IV | 2 mL | D42 |
| 8 | 10 | 3 | Circo | kPTV | IM | 2 mL | IN | 2 mL | IV | 2 mL | D42 |
| | | | kPTV | | | | | | | | |
| 9 | 4 | 1 | — | — | — | — | — | — | — | — | D0, |
| | | | | | | | | | | | D28, |
| | | | | | | | | | | | D42 |

TABLE 17
Description of challenge material and vaccines

| Product | Description |
|---|---|
| Negative Control | Sterile Diluent (water for injection) |
| Product (NCP) | |
| kPTV vaccine | Killed PTV, BEI inactivated, IF Adj, 6.5 log/ml |
| PCV2 vaccine | Porcine Circovirus Vaccine Type 2, killed baculovirus |
| | vector (Ingelvac CircoFLEX ®) |
| PRRSV vaccine | Porcine Reproductive and Respiratory Syndrome |
| | Vaccine modified live virus (Ingelvac ® |
| | PRRSV MLV) |
| PRRSV | PRRSV sw022208-972-1 propagated in AKMA104 |
| challenge | cells and diluted in MEM with 4% FBS to a titer |
| | of 4.5 log₁₀ TCID₅₀/mL |
| PTV challenge | PTV sw022208-6137A-1 LD pass 4 propagated in PK- |
| | WRL cells and diluted in MEM with 4% FBS to a |
| | titer of 4.5 log₁₀ TCID₅₀/mL |
| PCV2 challenge | PCV2-sw022208-194-8 pass 2 propagated in VIDO-R1 |
| | cells and diluted in MEM with 4% FBS to a titer of 4.0 |
| | log₁₀ TCID₅₀/mL |

### Animal and Housing Information

Information on the specific conditions of the animals used in the study are provided in Table 18. The pigs were transferred to the research housing facility between Day 21 and D28 and housed in four rooms. Prior to the start of the study, a veterinarian conducted a Health Examination and only allowed healthy animals to be included in the study. After the start of the study, pigs were eliminated only in the case of injury, illness (other than challenge-related) or death that would interfere with the outcome of the study. Prior to challenge, a necropsy was performed on all pigs removed for illness or found dead to determine the cause of illness or death.

TABLE 18
Animal Description

| Specifications | Requirements |
|---|---|
| Species: | Porcine |
| Breed: | Commercial mix |
| Age: | 21 ± 3 days of age on D0 |
| Weight Range: | Normal weight range for Cesarean-derived, |
| | colostrum-deprived (CDCD) pigs of this age |
| Source: | CDCD pigs from commercially sourced sows |
| Ownership: | BIVI |
| Sex: | Either females and/or males |
| Number: | Approximately 90 |
| Identification: | Ear tag (uniquely numbered) |
| Conditioning: | Pigs will be housed in individual incubators |
| | (A boxes) for at least 10 days post cesarean and then, |
| | prior to D0, moved to Brooders (B boxes) to acclimate |
| | with pen-mates. |
| Physiological | All piglets must be healthy at the time of |
| status: | vaccination and of challenge as determined |
| | by observation. |
| Serological status: | Seronegative for PRRSV; PCV2 titer <1:100 |

### Sample or Data Collection

### Necropsy and Tissue Collection

A necropsy was performed on any pig that died or was euthanized between challenge and scheduled necropsy. Selected pigs from group 9 were necropsied on D0 (3 pigs), D28 (3 pigs), and D42 (4-pigs). All remaining pigs were euthanized, necopsied and tissues collected on D42. At necropsy, the lungs were removed from the thoracic cavity, and the Investigator diagnosed each individual lung lobe as a percentage of lung with lesions and will provide an overall score for severity (normal, mild, moderate, severe).

### Gross Lung Lesions

Total lung lesions per animal were calculated using the following formula: total lung lesions=RA(0.11)+RC(0.10)+RD(0.34)+LA(0.05)+LC(0.06)+LD(0.29)+I(0.05) where RA—Right Apical=11%, RC—Right Cardiac=10%, RD—Right Diaphragmatic=34%, LA—Left Apical=5%, LC—Left Cardiac=6%, LD—Left Diaphragmatic=29%, I—Intermediate=5% (Weighting percentages used in the European Pharmacopoeia Monograph 2448). The pig was considered the experimental unit for all analyses. A one-way ANOVA was performed to assess significant differences between treatment groups. For the model, the treatment group was the fixed, independent variable and the continuous data (total lung lesion score) was the dependent variable. A difference was considered significant if the P<0.05. If a significant difference was noted in the one-way ANOVA, pairwise comparisons were made between groups using a Tukey-Kramer correction for comparison of multiple tests. Stastical analysis were done using the statistical software JMP (JMP 8.0.1; SAS, Cary, N.C.).

### Results

### Gross Lung Lesions

Group mean and standard error lung lesion scores are illustrated in FIG. 17. Group mean and standard error (bar) of the percent macroscopic lung lesions are shown for each group (1-9). Group vaccination information is given within the bar in the graph where No Vx=no vaccine was given; kPTV=killed PTV vaccine was administered; PCV2=PCV2 vaccine was administered. Group challenge information is provided on the x-axis.

### Conclusions

Macroscopic lung lesions were not observed in any of the animals in the strict negative control group (group 9). Based on a comparison of animals challenged with PRRSV compared to animals challenged with PRRSV and PTV; lung lesions were numerically exacerbated by PTV. However, when pigs were vaccinated with PTV, then challenged with PTV and PRRSV, there was no numerical reduction in lung lesions following vaccination. Interestingly, lung lesions were numerically worse in the PTV vaccinated animals challenged with PRRSV and PTV in comparison to non-vaccinated animals. When lung lesions were compared between non-vaccinated animals challenged with either PRRSV and PCV or PRRSV, PCV2, and PTV, lesions were numerically more severe in the triple challenged animals. Similarly, lesions were worse in triple challenged animals in comparison to animals challenged with PRRSV and PCV2 (non-vaccinated animals). When triple challenged animals were vaccinated for PTV, lesions were significantly reduced. When triple challenged animals were vaccinated for PCV2, lesions were numerically reduced. When triple challenged animals were vaccinated for both PCV2 and PTV, lesions were numerically reduced; however, were numerically higher than lesions in animals vaccinated with PTV alone.

### Example 5

### Objective

(1) The objective of this study was to evaluate a mixed challenge model of PCV2, PRRSV and PTV in CDCD pigs vaccinated with PTV, PCV2 and/or PRRSV.

### Materials and Methods

Experimental Design For the experiment, 95 CDCD pigs were randomly allocated into 10 treatment groups as indicated in Table 19. Treatment groups were either vaccinated or non-vaccinated and either challenged singularly or in combinations with PRRSV, PCV2 and PTV (groups 10-18) or left as negative controls (group 19). Table 20 provides the description of challenge materials and vaccines used in the study. All animals were observed daily for abnormal clinical signs; rectal temperatures were taken daily. All animals were weighed and bled on study day 0, 28, 35 and 42. The study was terminated at day 42.

TABLE 19
Study Design

| | | | | Challenge | | | | | | | | |
|---|---|---|---|---|---|---|---|---|---|---|---|---|
| | | | | PRRSV | | | PCV2 | | | PTV | | |
| | | Vaccination | | 4.5 log | | | 4.0 log¹⁰ | | | 6.5 log¹⁰ | | |
| | | | D14 | TCID50/ml | | | TCID₅₀/mL | | | TCID₅₀/mL. | | |
| | | | Lt | | Dose | | | Dose | | | Dose | |
| Grp | n | Day 0 Rt side | side | Route | size | Day | Route | size | Day | Route | size | Day |
| 10 | 10 | PRRS | WFI | IN | 2 ml | 28 | — | — | — | IV | 2 ml | 35 |
| 11 | 10 | WFI | WFI | IN | 2 ml | 28 | IN | 2 ml | 28 | IV | 2 ml | 35 |
| 12 | 10 | PRRS | WFI | IN | 2 ml | 28 | IN | 2 ml | 28 | IV | 2 ml | 35 |
| 13 | 10 | PRRS, PCV | WFI | IN | 2 ml | 28 | IN | 2 ml | 28 | IV | 2 ml | 35 |
| 14 | 10 | PRRS, PCV, PTV | PTV | IN | 2 ml | 28 | IN | 2 ml | 28 | IV | 2 ml | 35 |
| 15 | 10 | WFI | WFI | IN | 2 ml | 35 | IN | 2 ml | 35 | IV | 2 ml | 28 |
| 16 | 10 | WFI | WFI | IN | 2 ml | 28 | IN | 2 ml | 28 | IV | 2 ml | 28 |
| 17 | 10 | PRRS, PCV, PTV | PTV | IN | 2 ml | 35 | IN | 2 ml | 35 | IV | 2 ml | 28 |
| 18 | 10 | PRRS, PCV, PTV | PTV | IN | 2 ml | 28 | IN | 2 ml | 28 | IV | 2 ml | 28 |
| 19 | 10 | WFI | WFI | — | — | — | — | — | — | — | — | — |

TABLE 20
Description of Challenge material and Vaccines

| Product | Description |
|---|---|
| Negative Control | Sterile Diluent (water for injection) |
| Product (WFI) | |
| kPTV vaccine | Killed PTV, BEI inactivated, Incomplete Fruend's |
| | Adjvant, 6.5 log/ml |
| PCV2 vaccine | Porcine Circovirus Vaccine Type 2, killed |
| | baculovirus vector (Ingelvac CircoFLEX ®) |
| PRRSV vaccine | Porcine Reproductive and Respiratory Syndrome |
| | Vaccine modified live virus (Ingelvac ® |
| | PRRSV MLV) |
| PRRSV challenge | PRRSV sw022208-972-1 propagated in |
| | AKMA104 cells and diluted in MEM with 4% FBS to |
| | a titer of 4.5 log10 TCID50/mL |
| PTV challenge | PTV sw022208-6137A-1 LD pass 4 propagated |
| | in PK-WRL cells and diluted in MEM with 4% FBS |
| | to a titer of 4.5 log10 TCID50/mL |
| PCV2 challenge | PCV2-sw022208-194-8 pass 2 propagated |
| | in VIDO-R1 cells and diluted in MEM with 4% FBS |
| | to a titer of 4.0 log10 TCID50/mL |

### Animal and Housing Information

Information on the specific conditions of the animals used in the study are provided in Table 21. The pigs were transferred to the research housing facility between Day 21 and D28 and housed in four rooms. Prior to the start of the study, a veterinarian conducted a Health Examination and only allowed healthy animals to be included in the study. After the start of the study, pigs were eliminated only in the case of injury, illness (other than challenge-related) or death that would interfere with the outcome of the study. Prior to challenge, a necropsy was performed on all pigs removed for illness or found dead to determine the cause of illness or death.

TABLE 21
Animal Description

| Specifications | Requirements |
|---|---|
| Species: | Porcine |
| Breed: | Commercial mix |
| Age: | 28 ± 3 days of age on D0 |
| Weight Range: | Normal weight range for Cesarean-derived, |
| | colostrum-deprived (CDCD) pigs of this age |
| Source: | CDCD pigs from commercially sourced sows |
| Ownership: | BIVI |
| Sex: | Either females and/or males |
| Number: | 95 |
| Identification: | Ear tag (uniquely numbered) |
| Conditioning: | Pigs will be housed in individual incubators (A boxes) |
| | for at least 10 days post cesarean and then, prior to D0, |
| | moved to Brooders (B boxes) to acclimate with |
| | pen-mates. |
| Physiological | All piglets must be healthy at the time of vaccination |
| status: | and of challenge as determined by observation. |
| Serological status: | Seronegative for PRRSV; PCV2 titer <1:100 |

### Sample or Data Collection

### Necropsy and Tissue Collection

A necropsy was performed on any pig that dies or is euthanized between challenge and scheduled necropsy. Selected pigs from group 18 were necropsied on D0 (2 pigs), D28 (3 pigs), D35 (3 pigs), and D42 (4-pigs). All remaining pigs were euthanized, necopsied and tissues collected on D42. At necropsy, the lungs will be removed from the thoracic cavity, and the Investigator will diagnose each individual lung lobe as a percentage of lung with lesions and will give an overall score for severity (normal, mild, moderate, severe). After gross assessment, bronchoalveolar lavage was collected using sterile MEM.

### Gross Lung Lesions

Total lung lesions per animal were calculated using the following formula: total lung lesions=RA(0.11)+RC(0.10)+RD(0.34)+LA(0.05)+LC(0.06)+LD(0.29)+I(0.05) where RA—Right Apical=11%, RC—Right Cardiac=10%, RD—Right Diaphragmatic=34%, LA—Left Apical=5%, LC—Left Cardiac=6%, LD—Left Diaphragmatic=29%, I—Intermediate=5% (Weighting percentages used in the European Pharmacopoeia Monograph 2448). The pig was considered the experimental unit for all analyses. A one-way ANOVA was performed to assess significant differences between treatment groups. For the model, the treatment group was the fixed, independent variable and the continuous data (total lung lesion score) was the dependent variable. A difference was considered significant if the P<0.05. If a significant difference was noted in the one-way ANOVA, pairwise comparisons were made between groups using a Tukey-Kramer correction for comparison of multiple tests. Stastical analysis were done using the statistical software JMP (JMP 8.0.1; SAS, Cary, N.C.).

### Results

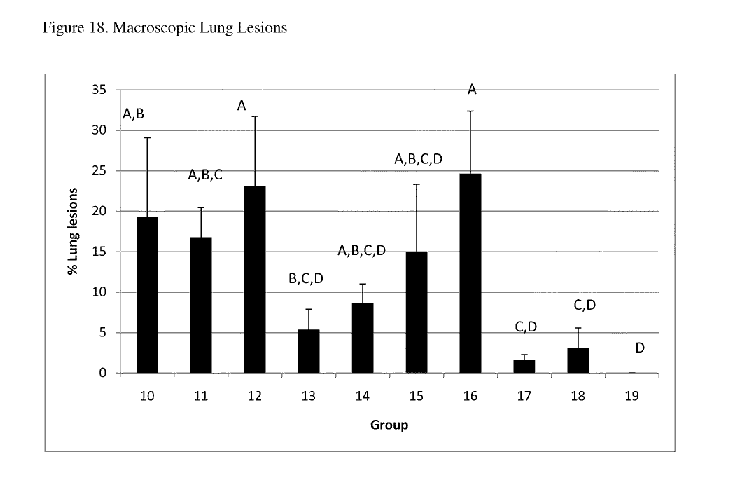

Gross lung lesions: Group mean and standard error lung lesion scores are presented in FIG. 18. Animals that were challenged with PRRSV, PCV2 and PTV at the same time (group 16) had the numerically highest lung lesion scores. Group mean and standard error (bars) for percent lung lesions were noted at the time of necropsy. Columns without a common superscript letter indicates statistically significant (p<0.05) differences between groups (non-adjusted t-test).

### Conclusions

In comparison to triple challenged animals in group 16, there was a significant reduction in lung lesions when animals were vaccinated with PRRSV, PCV2 and PTV (group 18). However, while there were numerically lower lesions in the triple vaccinated animals (group 18), there was not a significant reduction in lesions compared to animals vaccinated with PRRSV and PCV2 (group 13). Both vaccination with PRRSV and PCV2 (group 13) and vaccination with PRRSV, PCV2 and PTV (group 18) had significantly fewer lesions in comparison to pigs vaccinated with PRRSV alone (group 12). In conclusion, the data supports a significant reduction in lung lesions when animals are vaccinated for PRRSV, PTV and PCV2 but does not support that the reduction is greater than vaccination with PRRSV and PCV2 alone.

1. An immunogenic composition comprising
a. one or more porcine teschovirus antigens;
b. at least one immunogenic component effective against another disease-causing organism other than porcine teschovirus; and
c. a pharmaceutically acceptable carrier.

2. The composition of claim 1, wherein said at least one immunogenic component is an antigen of a pathogen selected from the group consisting of: Actinobacillus pleuropneumonia; Haemophilus parasuis, preferably subtypes 1, 7 and 14; Mycoplasma hyopneumoniae (M. hyo.); Porcine circovirus-2 (PCV-2); Porcine Reproductive and Respiratory Syndrome (PRRS) Virus; Reovirus; Swine Influenza Virus (SIV), and combinations thereof.

3. The composition of claim 1, wherein the amount of (a) and the amount of (c) together constitute an amount that is effective for increasing an immune response to an antigen upon administration to a subject in need of immunotherapy.

4. The composition of claim 1, wherein said one or more antigens of porcine teschovirus are selected from the group consisting of attenuated porcine teschovirus, inactivated porcine teschovirus, an immunogenic subunit of porcine teschovirus, a plasmid containing porcine teschovirus DNA sequences therein, and combinations thereof.

5. The composition of claim 1, wherein said antigen of said at least one immunogenic component other than porcine teschovirus is selected from the group consisting of an antigen that is attenuated, inactivated, an immunogenic subunit(s), a plasmid(s) containing DNA sequences coding for said antigen, and combinations thereof.

6. The composition of claim 1, wherein said at least one immunogenic component comprises an antigen from PCV2, PRRSV or a combination thereof.

7. The composition of claim 6, wherein said antigen from PCV2 is selected from the group consisting of attenuated PCV2, inactivated PCV2, an immunogenic subunit of PCV2, a plasmid containing PCV2 DNA sequences therein, and combinations thereof.

8. The composition of claim 6, wherein said antigen from PRRSV is selected from the group consisting of attenuated PRRSV, inactivated PRRSV, an immunogenic subunit of PRRSV, a plasmid containing PRRSV DNA sequences therein, and combinations thereof.

9. The composition of claim 6, wherein said PCV2 antigen is ORF2 protein or an immunogenic fragment thereof.

10. The composition of claim 6, wherein said PRRSV antigen is selected from the group consisting of an antigen of Ingelvac PRRS MLV vaccine, Ingelvac PRRS ATP vaccine, Ingelvac PRRS KV, and combinations thereof.

11. The composition of claim 1, wherein said pharmaceutically acceptable carrier is selected from the group consisting of solvents, dispersion media, coatings, stabilizing agents, diluents, preservatives, antibacterial and antifungal agents, isotonic agents, adsorption delaying agents, adjuvants, immune stimulants, and combinations thereof.

12. The composition of claim 11, wherein said adjuvant is selected from the group consisting of aluminum hydroxide, aluminum phosphate, saponins, water-in-oil emulsion, oil-in-water emulsion, water-in-oil-in-water emulsion, polymers of acrylic or methacrylic acid, copolymers of maleic anhydride and alkenyl derivative, the RIBI adjuvant system, Block co-polymerd, SAF-M, monophosphoryl lipid A, Avridine lipid-amine, heat-labile enterotoxin from E. coli (recombinant or otherwise), cholera toxin, IMS 1314, muramyl dipeptide, and combinations thereof.

13. A method of treating or preventing porcine respiratory disease complex (PRDC) or post-weaning multisystemic wasting syndrome (PMWS) in a subject in need thereof, the method comprising administering to the subject a therapeutically effective amount of an immunogenic composition comprising one or more porcine teschovirus antigens and a pharmaceutically acceptable carrier

14. The method of claim 13, wherein the subject exhibits one or more clinical signs associated with PRDC or PMWS.

15. The method of claim 13, wherein the clinical signs are associated with PCV2-infection.

16. The method of claim 13, wherein the clinical signs are associated with PRRSV infection.

17. The method of claim 13, wherein the clinical signs are associated with Mycoplasma hyopneumoniae infection.

18. The method of claim 13, wherein said immunogenic composition is administered using a method selected from the group consisting of intradermal, intratracheal, intravaginal, intramuscular, intranasal, intravenous, direct injection into target tissues, intraarterial, intraperitoneal, oral, intrathecal, subcutaneous, intracutaneous, intracardial, intralobal, intramedullar, intrapulmonary, and combinations thereof.

19. The method of claim 13, wherein said administration of porcine teschovirus antigen reduces the incidence of or severity of the one or more clinical signs.

20. The method of claim 13, further comprising administering to the subject at least one immunogenic component effective against a pathogen other than porcine teschovirus.

21. The method of claim 20, wherein the immunogenic composition further comprises the at least one immunogenic component effective against a pathogen other than porcine teschovirus.

22. The method of claim 20 or 21, wherein said administration of porcine teschovirus antigen and the at least one immunogenic component effective against a pathogen other than porcine teschovirus reduces the incidence of or severity of clinical signs of the other pathogen to a greater extent than administration of the immunogenic component administered in the absence of administration of porcine teschovirus antigen.

23. The method of claim 20 or 21, wherein the at least one immunogenic component is effective against a pathogen other than porcine teschovirus that is selected from the group consisting of Actinobacillus pleuropneumonia; Haemophilus parasuis, preferably subtypes 1, 7 and 14; Mycoplasma hyopneumoniae (M. hyo); Porcine circovirus-2 (PCV-2); Porcine Reproductive and Respiratory Syndrome (PRRS) Virus; Reovirus; Swine Influenza Virus (SIV), and combinations thereof.

24. The method of claim 20 or 21, wherein the pathogen other than porcine teschovirus is PCV-2, PRRSV or M. hyo.

25. The method of claim 13, wherein the subject is a mammal.

26. The method of claim 25, wherein the mammal is swine.

27. The method of claim 13, wherein the pharmaceutically acceptable carrier is selected from the group consisting of solvents, dispersion media, coatings, stabilizing agents, diluents, preservatives, antibacterial and antifungal agents, isotonic agents, adsorption delaying agents, adjuvants, immune stimulants, and combinations thereof.

28. The method of claim 13, wherein the one or more antigens of porcine teschovirus are selected from the group consisting of attenuated porcine teschovirus, inactivated porcine teschovirus, an immunogenic subunit of porcine teschovirus, a plasmid containing porcine teschovirus DNA sequences therein, and combinations thereof.

29. A method of producing an immunogenic composition comprising:
a. providing at least one porcine teschovirus antigen;
b. providing at least one immunogenic component effective against another disease-causing organism other than porcine teschovirus; and
c. combining (a) and (b) with a pharmaceutically acceptable carrier.

30. The method of claim 29, further comprising providing an adjuvant and combining the adjuvant with (a), (b) and the pharmaceutically acceptable carrier.

31. The method of claim 29, wherein said pharmaceutically acceptable carrier is selected from the group consisting of solvents, dispersion media, coatings, stabilizing agents, diluents, preservatives, antibacterial and antifungal agents, isotonic agents, adsorption delaying agents, adjuvants, immune stimulants, and combinations thereof.

32. The method of claim 29, wherein said at least one antigen of porcine teschovirus is selected from the group consisting of attenuated porcine teschovirus, inactivated porcine teschovirus, an immunogenic subunit of porcine teschovirus, a plasmid containing porcine teschovirus DNA sequences therein, and combinations thereof.

33. The method of claim 29, wherein the antigen of the at least one immunogenic component other than porcine teschovirus is selected from the group consisting of an antigen that is attenuated, inactivated, an immunogenic subunit(s), a plasmid(s) containing DNA sequences coding for said antigen, and combinations thereof.

34. The method of claim 29, wherein the at least one immunogenic component is effective against a pathogen other than porcine teschovirus that is selected from the group consisting of Actinobacillus pleuropneumonia; Haemophilus parasuis, preferably subtypes 1, 7 and 14; Mycoplasma hyopneumoniae (M. hyo); Porcine circovirus-2 (PCV-2); Porcine Reproductive and Respiratory Syndrome (PRRS) Virus; Reovirus; Swine Influenza Virus (SIV), and combinations thereof.

35. A method of reducing the incidence of or severity in a subject of one or more clinical signs associated with porcine respiratory disease complex or postweaning multisystem wasting syndrome, the method comprising the step of administering the immunogenic composition of claim 1 or claim 2 to a subject in need thereof, wherein the reduction of the incidence of or the severity of the one or more clinical signs is relative to a subject not receiving the immunogenic composition.

36. The method of claim 35, wherein the one or more clinical signs are selected from the group consisting of: respiratory distress, labored breathing, coughing, sneezing, rhinitis, tachypnea, dyspnea, pneumonia, red/blue discolouration of the ears and vulva, jaundice, lymphocytic infiltrates, lymphadenopathy, hepatitis, nephritis, anorexia, fever, lethargy, agalatica, diarrhea, nasal extrudate, conjunctivitis, progressive weight loss, reduced weight gain, paleness of the skin, gastric ulcers, macroscopic and microscopic lesions on organs and tissues, lymphoid lesions, mortality, and combinations thereof.

37. The method of claim 35, wherein the pharmaceutically acceptable carrier is selected from the group consisting of solvents, dispersion media, coatings, stabilizing agents, diluents, preservatives, antibacterial and antifungal agents, isotonic agents, adsorption delaying agents, adjuvants, immune stimulants, and combinations thereof.

38. The method of claim 35, wherein the at least one immunogenic component is an antigen of a pathogen selected from the group consisting of: Actinobacillus pleuropneumonia; Haemophilus parasuis, preferably subtypes 1, 7 and 14; Mycoplasma hyopneumoniae (M. hyo); Porcine circovirus-2 (PCV-2); Porcine Reproductive and Respiratory Syndrome (PRRS) Virus; Reovirus; Swine Influenza Virus (SIV), and combinations thereof.

39. The method of claim 35, wherein said one or more antigens of porcine teschovirus are selected from the group consisting of attenuated porcine teschovirus, inactivated porcine teschovirus, an immunogenic subunit of porcine teschovirus, a plasmid containing porcine teschovirus DNA sequences therein, and combinations thereof.

40. The method of claim 35, wherein said immunogenic composition is administered using a method selected from the group consisting of intradermal, intratracheal, intravaginal, intramuscular, intranasal, intravenous, direct injection into target tissues, intraarterial, intraperitoneal, oral, intrathecal, subcutaneous, intracutaneous, intracardial, intralobal, intramedullar, intrapulmonary, and combinations thereof.

41. A kit comprising (i) one or more antigens of porcine teschovirus; (ii) at least one immunogenic component effective against another disease-causing organism other than porcine teschovirus; (iii) a pharmaceutically acceptable carrier; and (iv) a container for packaging said antigens and said pharmaceutically acceptable carrier.

42. The kit of claim 41, further comprising instructions for use of the kit.

43. The kit of claim 41, wherein said one or more antigens of porcine teschovirus are selected from the group consisting of attenuated porcine teschovirus, inactivated porcine teschovirus, an immunogenic subunit of porcine teschovirus, a plasmid containing porcine teschovirus DNA sequences therein, and combinations thereof.

44. The kit of claim 41, wherein the at least one immunogenic component other than porcine teschovirus is an antigen of a pathogen selected from the group consisting of: Actinobacillus pleuropneumonia; Haemophilus parasuis, preferably subtypes 1, 7 and 14; Mycoplasma hyopneumoniae (M. hyo); Porcine circovirus-2 (PCV-2); Porcine Reproductive and Respiratory Syndrome (PRRS) Virus; Reovirus; Swine Influenza Virus (SIV), and combinations thereof.

45. The kit of claim 41, wherein said pharmaceutically acceptable carrier is selected from the group consisting of solvents, dispersion media, coatings, stabilizing agents, diluents, preservatives, antibacterial and antifungal agents, isotonic agents, adsorption delaying agents, adjuvants, immune stimulants, and combinations thereof.

46. The kit of claim 41, wherein (i), (ii), (iii) and (iv) are packaged separately.

47. A method for evaluating the ability of an immunogenic composition to prevent or reduce the severity of PRDC or PMWS in a porcine subject, the method comprising:
a. administering to the subject a candidate immunogenic composition;
b. exposing the subject to a porcine teschovirus isolate in an amount sufficient to cause infection in an unvaccinated subject; and
c. monitoring the subject for one or more clinical signs of PRDC or PMWS, thereby evaluating the ability of the candidate immunogenic composition to prevent or reduce the severity of PRDC or PMWS.

48. The method of claim 47, wherein the candidate immunogenic composition comprises one or more porcine teschovirus antigens; at least one immunogenic component effective against another disease-causing organism other than porcine teschovirus; and a pharmaceutically acceptable carrier.

49. The method of claim 47, further comprising exposing the subject to PCV2, PRRSV or a combination thereof.

50. The method of claim 47, wherein the porcine subject is a young PTV-negative piglet, a barrier-raised specific pathogen-free piglet, or a caesarian-delivered piglet.


SEQUENCE LISTING (2 sequences)

SEQ ID NO: 1 - DNA, 7123 bp, Porcine teschovirus
  modified_base (172)..(172): a, c, t, g, unknown or other
cccccccccccccttccccctctggacttgtaactggtaagtgataatccttggaagctaggtactcgttacgcwagttttggattatcttgtgcccaacaytagwyytcggwctyytgttgtgtttaaacacagaaatytkrytryytggttatgaattcattggawyaanccctctgaaagacytgctctggcgcgagctaaagcgcaattgtcaccrggtattgcaccaatggtggcgacagggtacagaagagcaagtactcctgactgggyaatgggactgcattgcatatccctaggcacctattgagatttctctggggcccaccagcgtggagttcctgtatgggaatgcaggactggacttgtgctrcctgacagggtcgcggctggccgtctgtactttgtatagtcagttgaaaytcacyatggcatgtttgaaaattttctctctgaaacgyaargataagagccactcatattctcctcgtgrgattgagttgaartayaattcagattttgcyttyaaaccaagrccrttrgcaccmythctgcgycttgaaccatctgatrccaaracrcgcagraktgagtgygcwgadrttgaatatgattcwtggtacccaaacccyytdgayccaacytctytgatytgtgatktwgabcttgaaatgctvcarttccarggaactggrwcytctagratggaraatgghaatacyaataattctggyaayactggagtrathaattayaacttttactcaaaytcttacacagatgcaattgaycthtctggtgcratgtcatcvcaagaatcaaatgcwgctgaraatgctgcwtcagggcctncatcyytwytkaaagctggrataaatgctgctgctagnatnggacccttgcttgchgaaccaaattttgargaacataaratgtcagayagagtgganatbattcaraaaggaaccagcachattataacacagaatagygttggctcaaacacytacrcaagctgyccaaaatctgacatbachtcagtggctgaygagagcacancnggtggncctgctgtctctcgbttcnngacagtdaaagcccanccatggncnncagcnaartcngtttatgchtttcaagtagcnncatytccatatgcacttactgcccagaahacacchtttaaatctcttttgtcaaaacaycagcttatgaaatgytgytggattgtdcaagtrcaagtgaacacaaccagatttcatggtggttgtttgggtgtttttgcaattcctgagtttaatgtgtayaacaaattaaatggccttacaaatdactacactaayaattcagrtgggavagtgwctgayatttgggacagcttttccacmtggyayaayccagaagcaatgtatggtgchtggtattataavcatgtgtatgatgagacagabaaacaytggtacaaaccagaacctgagcmatatggyggvgtrtcmccccartccttgttttgctttccccaycagcttattaatcccagracaaattcgtcagcaacattrtgcbtgccttatgttgattgtgggccaataactgaygtgactgtvcattgcccgtgggccattgtgattgttgttttgagagaattgacagtggctagcggaggaacacctaacgttgacattaatgtctcagtggctccatgcgatgttgagttccatgggctgcgccagaattcaatgtaccaaggtcctataccaaaaatagacattgatgcaagtaaagcccttttctcctcaacacagccttacccagctccacccgtgtacgggagaataccagcaagcagccctgcttttatgcctggagaatttcttgattatgtacaacttgcccaaatcccatcccttgtgaagaaccgtgaaatacgtgttactaatacagacaaggctgaccccatattttgcatgccattgaggcctactgatacagaggccatgaataccatgttgggctctgtgacccgaatgttttcacagtggcgtgggtccatagtaataaggtgtgtttttgtgggcaatcagatgcaaaactgcaggattcttttggcatggacacccccgggttttggggcaactcacccaaccaaaatggaagatgcaatgcagggccactatgtaatttatgatactgggattgatagtagtgctgatttagtaattccttatgtggctccctatgactttactccagttagaacaccctacacaaaaaccgaactaccaccaactggcaatagccaggtcttagttaacacaccatactggtcccgaggattcataaccatatggcagtacacaaatttggcctccccccccggctcaccatcacaagcagattttctcctgtttgcttttgcaggagaagactatgtgctgaaagggccatccaacattgttgctggttaccagggaaatgagagttcacctctccaacctgctgagacaggctgtgatagggcaatgactaataatgcatcaaatgaaaaccccattccccttgcagtggaagccattggctcatcaaaagtagatttcttttgggacaggtattttcacatcgtgacaattccaggagtatccaacatagacccatactacataaggttgaatatttcagatatcttgtccgatccagtgatcaggcaatctttacatgccacctatttgagatgtggaatctctgttgcggtgagggcaatgcctgcagacccctctagcacagaatgcttggatggtttgacaattaacatgctttatgtcccaccaggcagctcgtgggccatagataaaatagaccagaagatgcctatagagcaagcacaagattcaactgggaattacgcactaccttccttcacgtggaagcccaatcaaacacctgtgtttacgtgctcaattccttatgtgtcctttacaacagtattaccaacagcatactctggttttgaaacgacggtttcaattccaaagaggaataaccaaataccgcaagatttcggatttggaatgcttgttttacggccgtcaatgccccchrcacgnaaattggtdatatcagcatgggttaaattyaahaatgtraggctattttgyccacgtcctgggatacatgatgaagcatcytaytcaaacaratctachhcaattccaggrrcwgdracargncrrctbtcadctrcmatgacyacbatgdcattbcaggggccaggtgctacvaacttttctytgytgaagcaagcaggdgatrtwgaagaaaaycccgggccttccctttcyaaattgtaycarattttgaargayccagcagttgatgccctttgtgaagcatatgatgaattgaagaaatttaaagarcaagcyacyaayytkttrgattcwttytctggagawtctgagaayccwtggctdaayaaatttrttaartatttrggctaygccatyytrgcytggaaatcmcttcatgaycchatgacagcagcrgcwgtcksytttataattggtagtgatgtgacagcatttgtwgtttcyaaattagccaarcayytgaagaaatttgchaaracygayccwcckcctgtgcctaagcccagakcytgyaagaaagargggtgttgttgtggyaacaarcacaactaccctgatgarctsaacccdttytcagagaryggsttytggagtcghtttaagaaargbcayytycaagghccmatgcargaygtgagctctttgattaayatcytgaagggtgcagaatggatwtaccaccartttgagaarrtaatyaaatggctwaagacytggagaacwgctgartatgaggtytcytctgaytwtctccagaatgagatgaagaaytayccacaytatyacagaaagtayaaggaatgtgcaaayaaytacaggcacccagacagractgaagtrgcaramtayttcargaaratgagaaartgtgcwgcccatgttaayccaagattgatgrgcatgtttcctgaatttgacccwtctccwcctgatccyactcgyccygarccagttgtwgtcgtcttgcgcggcgcaccyggrcagggyaagagtgtgtgytctgagatgcttgcyanratgctttcytayacyttrgttggcaartcwtcttaytatgcattyaaytcagccacyaarcattttgayggctayaaacagcargcygtcacaatmattgatgatctygggcaggacacatcyggtgargatttccggctgttctgycagctggtrtcmacyactgartgtatygtrccyatggctgatttgccygataaaggratgcaytttaartctgaaatwytkattgccaayacyaacytgccttcttttaaccccattactgtgtctgatccagctgctatwaaragragaatttttcttgacytwacwgttgaagcaaaccgggcstacaccaagcccgatgggacwctwgayctygtccgagcsctycaascaactggyaargaatctcaatcyccwttgttgaggcargaccacaayattttktaykcmgactgygtygcmttyaagcatggcarryacaccttgtctttyctrgargtctttgacatgatyaagagagarctacagagrcgkcagaatgtttctaactctytgacraatctkttcactttccaaggaccmartgatgactcctggtttacmacyttytacaagaaatggaacttgagagcaaargaaaattctgaggaaaagctgattcttgarcttatyagataytgtcatggatctgakatgctraaagartattgycagcttgctgcwgcyaacaargaraagaaracagrytaytggaayatggtyaggattattgaagamatactygcagccctaaccytratyytgtcyctyatytccattatgatagttatgtaycagctctttttccagggrycctaygaggctactgcmayaaaaccmrcaaarccwaatagrcaatctctkttraagytagtkgaratgcaagggccaaaaggacaagccaatatggaaatggaragaacyttratgaaraagaacatagtggaratgacatatgagaagcacaacgggargtttcaaactaccacagtcctgtttgtgcgtgatcggatttttctgatcaacactcacattttgtcatcaattaagaacttccactatgagaacactgagattccagcagcytcagttcagaaagttcaggccatatttgatggacatycmtctgatgtractgcagtccarttyactgttggrcgrcaataycgtgacatcacaagcaayttyataawcagyytrccaaatcctggyacaccaatagttggcttgatgaagacwgaaggtagcagttayatctggtctggtgaawgyytsccatttaaraatacaatgaayacttatgaaggctgtgtycctcaygtkttaccatayaargccgtgacagcccayggytactgtggctctgtyrtggtcgcagatgctggagtytggaaaggcatttgtggcatacactcyytaggagatggtgcyataggtgcagcaacagttttgtcaagacagcatttgctyaaycttcttgagggattyytrgaattycagggaaaaatttatgatgtgcagaaarcagaytttgtgtacacaccyactagaacctctttgaagccaacatttrtgtgtgttgayccaaaattrgaacctgcagctctttcacatytagacccaaggytgaaagarccmgaraattttaaagctgtyattttrtccaarcatgtgggcgaytcactgcarctyccatggggcattcgatgggcctcatacaagtatgcagagagrattcgygccatgttrccaccagacttyttrgaaccyctttctgttagggaagcagttgaaggaattgatggmytrgacccaatggacatggayaaatcmcctggmcttccataygttaagaaaggcttgcgccggactgacytrtggaayccmaagactggwtcttcwttggagctgatggctgagataaaymgatatctygattacaatnatgayaaacatgtgtttctgacatttttraargatgaaytgagaccaaaagagaaagtycaagccgggaagacccgygtcatwgatgtggcagggtttggrcatgcwatagtyggccgratgytrttyggtcggytstttgcyttctttcacaagaatcccggytggaatacaggatcagctgttggagtwaatccagayttggcctggacacaratcttttayactgcycctagccgsaatgtrytggctatggaytaytctggatttgatgcwtcwcayacwtctggcatgttttgcattttgaagcattttytraccacycttggttatggractttacagctwtcctatatwgattcyctctgttattcyaaacaccaytgggatgatgaaacctaycgccttgayggwggattrccytctggrtgctctggcacracaatttttaayaccattatgaacaacattgttgcccgagctgcmgcmagttaygctgcygatggccctgttggcatcctctgttatggwgatgacatyttrgtttcatccccwgagaaattyccagtytctgactggttggagttttactccaagacaccttayaaagtgactgcagcygataagagtgagcagatygactggcgtgacatcacacagtgcacgttccttaaacggggatttgtgctggatggtagtctygtgcgmccagtyatggaagaacaacatttggctgaattgctraaatgggctagrccaggaacattgcaggccaagttgctgtcyattgcccagttggcatttcatcttccgcgcycggcrtayaatcgcctyatgctcccrtttgargaggctggatatgaaatccccagccatgaacgcctgaatgaggartggcgcgaratgttcatttgaatrgctacagttgtgagcagtttagtgattttctgcctgttgcttaagtagtttagttgagcccaaaaaaa

SEQ ID NO: 2 - DNA, 7159 bp, Porcine teschovirus
tacgcacggccggtacagtgaaactgcgaatggctcattaaatcagttatggttcctttggatttgtaactggtaagtgatgaaccttggaagctaggtacttgttacgctagttttggattgtcttgtgcccaacattagtcttcggacttctgttgtgtttaaacacagaaatttgaatatctgactacgaattcactggatcaacccttttgaaagacctgctctggcgcgagctaaagcgcaattgtcaccaggtattgcaccaatggtggcgacagggtacagaagagcaagtactcctgactgggtaatgggactgcattgcatatccctaggcacctattgagatttctctggggcccaccagcgtggagttcctgtatgggaatgcaggactggacttgtgctgcctgacagggtcgcggctggccgtctgtactttgtatagtcagttgaaactcactatggcatgtttgaaaattttctctctgaaacgtaaggataagagccactcatattctcctcgtgagattgagttgaagtataattcagattttgccttcaaaccaaggccgttggcacccctcctgcgtcttgaaccatctgataccaaaacacgcagaattgagtgcgcagatattgaatatgattcttggtacccaaacccccttgatccaacctctctgatttgtgatgtagatcttgaaatgctccagttccagggaactggatcctctaggatggaaaatggaaatactaataattctggcaatactggagtgattaattacaacttttactcaaactcttacacagatgcaattgatctctctggtgcgatgtcatcacaagaatcaaatgctgctgaaaatgctgcatcaggaccttcatctttattgaaagctggaataaatgctgctgctaggattggacccttgcttgcagaaccaaattttgaggaacataaaatgtcagatagagtggatattattcaaaaaggaaccagcactattataacacagaatagcgttggctcaaacacttacacaagctgtccaaaatctgacattacctcagtggctgacgagagcacaactggtggacctgctgtctctcgtttcgtgacagtaaaagcccagccatggacaccggcaaagtctgtttatgcctttcaagtagcacatttaccatatgcacttactgcccagaatacaccctttaaatctcttttgtcaaaacatcagcttatgaaatgctgctggattgtacaagtgcaagtgaacacaaccagatttcatggtggttgtttgggtgtttttgcaattcctgagtttaatgtgtacaacaaattaaatggccttacaaataactacactaataattcagatgggagagtgactgatatttgggacagcttttccacatggcataacccagaagcaatgtatggtgcctggtattataagcatgtgtatgatgagacagataaacactggtacaaaccagaacctgagcaatatggcggggtgtccccccagtccttgttttgctttccccaccagcttattaatcccaggacaaattcgtcagcaacattgtgcctgccttatgttgattgtgggccaataactgacgtgactgtacattgcccgtgggccattgtgattgttgttttgagagaattgacagtggctagcggaggaacacctaacgttgacattaatgtctcagtggctccatgcgatgttgagttccatgggctgcgccagaattcaatgtaccaaggtcctataccaaaaatagacattgatgcaagtaaagcccttttctcctcaacacagccttacccagctccacccgtgtacgggagaataccagcaagcagccctgcttttatgcctggagaatttcttgattatgtacaacttgcccaaatcccatcccttgtgaagaaccgtgaaatacgtgttactaatacagacaaggctgaccccatattttgcatgccattgaggcctactgatacagaggccatgaataccatgttgggctctgtgacccgaatgttttcacagtggcgtgggtccatagtaataaggtgtgtttttgtgggcaatcagatgcaaaactgcaggattcttttggcatggacacccccgggttttggggcaactcacccaaccaaaatggaagatgcaatgcagggccactatgtaatttatgatactgggattgatagtagtgctgatttagtaattccttatgtggctccctatgactttactccagttagaacaccctacacaaaaaccgaactaccaccaactggcaatagccaggtcttagttaacacaccatactggtcccgaggattcataaccatatggcagtacacaaatttggcctccccccccggctcaccatcacaagcagattttctcctgtttgcttttgcaggagaagactatgtgctgaaagggccatccaacattgttgctggttaccagggaaatgagagttcacctctccaacctgctgagacaggctgtgatagggcaatgactaataatgcatcaaatgaaaaccccattccccttgcagtggaggccattggctcatcaaaagtagatttcttttgggacaggtattttcacatcgtgacaattccaggagtatccaacatagacccatactacataaggttgaatatttcagatatcttgtccgatccagtgatcaggcaatctttacatgccacctatttgagatgtggaatctctgttgcggtgagggcaatgcctgcagacccctctagcacagaatgcttggatggtttgacaattaacatgctttatgtcccaccaggcagctcgtgggccatagataaaatagaccagaagatgcctatagagcaagcacaagattcaactgggaattacgcactaccttccttcacgtggaagcccaatcaaacacctgtgtttacgtgctcaattccttatgtgtcctttacaacagtattaccaacagcatactctggttttgaaacgacggtttcaattccaaagaggaataaccaaataccgcaagatttcggatttggaatgcttgttttacggccgtcaatgccccctacacggaaattggtgatatcagcatgggttaaatttaacaatgtgaggctattttgtccacgtcctgggatacatgatgaagcatcttactcaaacaaatctaccccaattccagggactgagacaggccaactgtcaactgcaatgactactatgtcattccaggggccaggtgctaccaacttttctctgctgaagcaagcaggggatgttgaagaaaatcccgggccttccctttccaaattgtatcagattttgaaggatccagctgttgatgccttgtgtgaagcatatgatgaattgaagaaatttaaagaacaagccaccaatttgttagattcattttctggagattctgagaatccttggctgaataaatttattaaatatttgggctatgccatcctagcctggaaatcacttcatgatcctatgacagcagcagctgtcgcttttataattggtagtgatgtgacagcatttgtagtttctaaattagccaaacacctgaagaaatttgcaaaaaccgaccctccgcctgtgcctaagcccagatcctgcaagaaagaggggtgttgttgtggtaacaaacacaactaccctgatgaactcaacccattttcagagaatgggttttggagtcgttttaagaaaggtcaccttcaaggacccatgcaggacgtgagctctttgattaatatcttgaagggtgcagaatggatataccaccagtttgagaagataattaaatggctaaagacctggagaacagctgaatatgaggtctcttctgattttctccagaatgagatgaagaattatccacactattacagaaagtacaaggaatgtgcaaacaattacaggcacccagacagaactgaagtggcagaatatttcaggaaaatgagaaagtgtgcagcccatgttaatccaagattgatgggcatgtttcctgaatttgacccttctcctcctgatcccactcgtcctgaaccagttgtagtcgtcttgcgcggcgcacccggacagggcaagagtgtgtgctctgagatgcttgctaggatgctttcctatactttggttggcaagtcttcttattatgcattcaattcagccactaagcattttgatggctataaacagcaggccgtcacaatcattgatgatctcgggcaggacacatctggtgaagatttccggctgttctgtcagctggtgtcaaccactgagtgtatcgtacctatggctgatttgcccgataaagggatgcattttaagtctgaaatacttattgccaataccaacctgccttcttttaaccccattactgtgtctgatccagctgctataaagaggagaatttttcttgacttaactgttgaagcaaatcgggcgtacaccaagcccgatgggacacttgaccttgtccgggcgcttcagccaactggcaaagaatcccaatctccgctattgaggcaagaccataatattctttatgctgactgtgttgcctttaaacatggcaagtacaccttgtcttttctagaggtctttgatatgattaagagagagctgcagaggcggcagaatgtttccaactctttgacaaatctctttactttccaagggcctagtgatgactcctggtttaccaccttttataagaaatggaacttgagagcaaaggaaaattctgaggaaaagctgattcttgaacttatcagatactgtcatggatctgatatgctaaaagagtattgtcagcttgctgcagctaacaaggaaaagaagacagactactggaatatggtcaggatcattgaagaaatacttgcagccctaaccttaatcctgtccctcatttccattatgatagttatgtaccagctctttttccagggaccctacgaggctactgccacaaaaccagcaaagccaaataggcaatctctgttgaagctagtggaaatgcaagggccaaaaggacaagctaacatggaaatggaaagaactttgatgaaaaagaacatagtggaaatgacatatgagaagcacaatgggaaattccaaaccaccactgtgatatttgtgcgtgatcggatttttctgatcaacactcacatcttaacatcaattaagaacttccactatgagaatactgagatcccagcagcttcagttcagaaagttcaggcgatttttgatggacacccctcagatgtgacagcagtccaatttactgttggacgacagtaccgtgacattaccaacaatttcattaacagtttgccaaaccctggtacaccaatagttggtttgatgaagacagaaggtagcagctatatgtggtctggtgaatgccttccattcaaaaatacaatgaacacctatgaaggttgtgtcccccatgtgttaccatacaaggctgtaacagcccatggctactgtggctctgttatggttgcagatgctggagtttggaaaggaatctgtggtatacactccttgggagatggtgccataggtgctgcgactgtgttgtcaagacagcatttgcttaaccttcttgagggattcctggaatttcaggggaaaatttatgatgtgcagaaagcggattttgtgtacacacctaccaggacttccttgaaaccaacatttgtgtgtgttgacccaaaattagaaccagcagctctctcacatttggaccctaggttgaaagaaccagaaaactttaaagctgttattttgtcaaaacatgtgggcgactcactgcagcttccatggggcatccgatgggcctcatataagtacgcagagaagattcgcgccatgttgccaccaaacttcttggaacccctttctattagggaagcagttgaaggaattgatggattagacccaatggacatggataaatcacctggacttccatatgtcaagaaaggcttgcgccggaccgacctctggaaccccaagactggaccttccttggagctgatggctgaggtaaatagatatcttgattataactatgataaacatgtgtttctgacatttttaaaagatgaattgagaccaaaagagaaagtccaagccgggaagacccgtgtcattgatgtggcagggtttgggcatgcaatagttggccgaatgttatttggtcgactgtttgcctttttccataagaatcccggttggaacacaggatcagctgttggtgtaaacccagatttggcctggacacagatcttttatactgctcctagccgcaatgtgctggctatggattactctggatttgatgcttcccatacatctggcatgttttgcattttaaagcactttttgaccactcttggttatggacctttgcagctatcctatatagattccctctgttattctaaacatcattgggatgatgaaacctaccgccttgatggtggattgccttctggatgctctggcacgacaattttcaataccattatgaataacattgttgcccgagctgcagctagttatgctgctgatggtcctgttggcatcctctgctacggtgatgacatcttggtttcatctcctgagaaatttccagtttctgactggttggagttttactccaagacaccctataaagtgactgcagccgataagagtgaacatattgactggcgcgatatcacacagtgcacgttccttaagcggggatttgtgctggatggtagtcttgtgcgaccagttatggaagaacaacatttagctgaattgctaaaatgggctaggccaggaacgttgcaggccaagttactgtccattgcccaattggcatttcaccttccgcgctcggcgtacgatcgcctcatgctcccatttgaggaggctggatatgaaattcccagccacgaacgcctgaatgaggagtggcgcgagatgttcatttgaatrgctacagttgtgagcagtttagtgattttctgcctgttgcttaagtagtttagttgagcccaaaaaaa